(19) United States
(12) Patent Application Publication
(10) Pub. No.: US 2009/0104164 A1
(43) Pub. Date: Apr. 23, 2009

(54) ANGIOGENIC CELLS FROM HUMAN PLACENTAL PERFUSATE

(75) Inventors: Xiaokui Zhang, Livingston, NJ (US); Mohammad A. Heidaran, Chatham, NJ (US); Vanessa R. Voskinarian-Berse, Madison, NJ (US); Lin Kang, Edison, NJ (US); Henry Rendon Barragan, Little Ferry, NJ (US)

Correspondence Address: JONES DAY, 222 EAST 41ST ST, NEW YORK, NY, 10017, US

(73) Assignee: Celgene Cellular Therapeutics, Warren, NJ (US)

(21) Appl. No.: 12/239,679

(22) Filed: Sep. 26, 2008

Related U.S. Application Data
(60) Provisional application No. 60/995,679, filed on Sep. 26, 2007.

(51) Int. Cl. A61K 35/12; C12N 5/06

(52) U.S. Cl. 424/93.7; 435/375

(57) ABSTRACT

Provided herein are the production of vasculogenic or angiogenic cells from placental perfusate. Also provided are methods of treating an individual having a cardiac or vascular insufficiency, disease, disorder or condition comprising administering to said individual placental perfusate, placental perfusate cells, or combinations of placental perfusate or perfusate cells with placental or non-placental hematopoietic stem cells or adherent placental stem cells.

This application claims benefit of U.S. Provisional Application No. 60/995,679, filed on Sep. 26, 2007, which is hereby incorporated by reference in its entirety.

### 1. FIELD

Provided herein are isolated placental perfusate, populations of placental perfusate cells, compositions comprising the perfusate or perfusate cells, and methods of using the placental perfusate or placental perfusate cells to produce angiogenic cells and angiogenic cell populations, and to treat individuals having a cardiac or vascular disease, disorder or insufficiency.

### 2. BACKGROUND

Human stem cells are totipotential or pluripotential precursor cells capable of generating a variety of mature human cell lineages. Evidence exists that demonstrates that stem cells can be employed to repopulate many, if not all, tissues and restore physiologic and anatomic functionality.

Many different types of mammalian stem cells have been characterized. See, e.g., Caplan et al., U.S. Pat. No. 5,486,359 (human mesenchymal stem cells); Boyse et al., U.S. Pat. No. 5,004,681 (fetal and neonatal hematopoietic stem and progenitor cells); Boyse et al., U.S. Pat. No. 5,192,553 (same); Beltrami et al., Cell 114(6):763-766 (2003) (cardiac stem cells); Forbes et al., J. Pathol. 197(4):510-518 (2002) (hepatic stem cells). Umbilical cord blood, and total nucleated cells derived from cord blood, have been used in transplants to restore, partially or fully, hematopoietic function in patients who have undergone ablative therapy. Placental perfusate comprises a collection of placental cells obtained by passage of a perfusion solution through the placental vasculature, and collection of the perfusion fluid from the vasculature, from the maternal surface of the placenta, or both. Methods of perfusing mammalian placentas are described, e.g., in U.S. Pat. No. 7,045,146 and U.S. Pat. No. 7,255,879. The population of placental cells obtained by perfusion is heterogenous, comprising, inter alia, CD34⁺ cells, nucleated cells such as granulocytes, monocytes and macrophages, a small percentage (less than 1%) of tissue culture substrate-adherent placental stem cells. No one to date has described the use of placental perfusate, or populations of placental cells from perfusate, in the production of angiogenic cells.

### 3. SUMMARY

Provided herein are methods of producing angiogenic or vasculogenic cells from placental perfusate or placental perfusate cells, e.g., total nucleated cells from placental perfusate.

In one aspect, provided herein is a method of producing angiogenic or vasculogenic cells, e.g., with the ability to form vasculature, comprising culturing placental perfusate or perfusate cells under conditions in which a plurality of said cells differentiate into cells of the vascular or cardiac system, e.g., into vascular cells, e.g., endothelial cells, or into cardiac cells. In a specific embodiment, said placental perfusate or said placental perfusate cells comprise hematopoietic placental stem cells, e.g., CD34⁺ placental cells. As used herein, the term “CD34⁺ placental cells” refers to CD34⁺ cells, e.g., endothelial progenitor cells, obtained from placenta and not from placental blood or umbilical cord blood. In another specific embodiment, said placental perfusate cells, e.g., said CD34⁺ placental stem cells produce amounts of one or more angiogenesis-related markers at a higher level than an equivalent number of CD34⁺ cells from umbilical cord blood. In specific embodiments, said markers comprise CD31, VEGF-R and/or CXCR4. In a specific embodiment, said placental CD34⁺ cells are CD45⁻. In a more specific embodiment, said CD34⁺, CD45⁻ cells produce amounts of one or more angiogenesis-related markers at a higher level than an equivalent number of CD34⁺ cells from umbilical cord blood. In specific embodiments, said markers comprise CD31, VEGF-R and/or CXCR4. In another specific embodiment, said culturing comprises contacting said perfusate cells, e.g., said CD34⁺ placental cells, with transforming growth factor-beta (TGF-β), fibroblast growth factor (FGF), plasminogen, tissue plasminogen activator (tPA) and one or more matrix metalloproteases. In a more specific embodiment, said culturing is for 18-24 hours. In another more specific embodiment, said cells form visible vessel structures after 24 hours of said contacting. In a more specific embodiment, said contacting is under conditions in which said cells produce visible vessel structures after 24 hours, and CD34⁺ stem cells from umbilical cord blood do not form visible vessel structures, or detectably fewer vessel structures than said perfusate cells or CD34⁺ placental cells. In another specific embodiment, said contacting is performed in vitro. In another specific embodiment, said contacting is performed in vivo. In a more specific embodiments, said in vivo contacting is performed in a mammal. In a more specific embodiment, said mammal is a human. In certain embodiments, any of the CD34⁺ cells described herein, or populations of CD34⁺ cells, are expanded.

In certain embodiments, provided herein is a method of forming vessels from a population of placental perfusate cells, comprising contacting said population of cells with conditions that promote the formation of vessels. In a specific embodiment, said population of placental perfusate cells is total nucleated cells from placental perfusate. In another specific embodiment, said contacting is performed in vitro. In another specific embodiment, said contacting is performed in vivo. In another specific embodiment, said population of placental perfusate cells comprises placental perfusate cells isolated from perfusion of a single placenta. In another specific embodiment, said placental perfusate cells are CD34⁺ cells. In a more specific embodiment, said CD34⁺ cells are CD34^{+CD}45⁻ cells. In a more specific embodiment, said CD34⁺ cells or CD34^{+CD}45⁻ cells express a higher level of at least one of CD31, CXCR4 or VEGFR than an equivalent number of CD34⁺ cells from umbilical cord blood. In another specific embodiment, said population of placental perfusate cells comprises isolated CD34⁺ cells not isolated from said perfusate (e.g., isolated from umbilical cord blood, placental blood, peripheral blood, bone marrow, or the like). In a more specific embodiment, said CD34⁺ cells are isolated from placenta. In another more specific embodiment, said CD34⁺ cells are isolated from umbilical cord blood, placental blood, peripheral blood, or bone marrow. In another more specific embodiment, said CD34⁺ cells express a higher level of CD31, CXCR4 or VEGFR than an equivalent number of CD34⁺ cells from umbilical cord blood. In another specific embodiment, said CD34⁺ cells are CD34⁺, CD45⁻ cells. In another more specific embodiment, said CD34⁺, CD45⁻ cells express a higher level of CD31, CXCR4 or VEGFR than an equivalent number of CD34⁺ cells from umbilical cord blood.

In another aspect, provided herein is a method for treating an individual having a cardiac or vascular insufficiency or defect, e.g., by promoting angiogenesis or angiogenesis in the individual, comprising administering to the individual placental perfusate or placental perfusate cells in an amount sufficient to produce a detectable improvement in, or reduction in the worsening of, one or more symptoms of the cardiac or vascular insufficiency. In a specific embodiment, the placental perfusate or placental perfusate cells are contained within an implantable or injectable composition. In another specific embodiment, the placental perfusate or placental perfusate cells are contained within a composition as provided herein. In another specific embodiment, the placental perfusate or placental perfusate cells are supplemented with a plurality of CD34⁺ placental cells, placental adherent cells, or both.

In another embodiment, provided herein is a method of treating an individual having a cardiac or vascular disease, disorder, condition or insufficiency, comprising administering human placental perfusate cells to said individual in an amount sufficient to treat said disease, disorder, condition or insufficiency. In a specific embodiment, said disease, disorder, condition or insufficiency is peripheral vascular disease, acute or chronic myocardial infarct, cardiomyopathy, congestive or chronic heart failure, cardiovascular ischemia, hypertensive pulmonary vascular disease, peripheral arterial disease, or rheumatic heart disease. In another specific embodiment, said placental perfusate cells are total nucleated cells from placental perfusate. In another specific embodiment, said population of placental perfusate cells comprises placental perfusate cells isolated from perfusion of a single placenta. In another specific embodiment, said placental perfusate cells are CD34⁺ cells. In a more specific embodiment, said CD34⁺ cells are CD34^{+CD}45⁻ cells. In a more specific embodiment, said CD34⁺ cells or CD34^{+CD}45⁻ cells express a higher level of at least one of CD31, CXCR4 or VEGFR than an equivalent number of CD34⁺ cells from umbilical cord blood. In another specific embodiment, said population of placental perfusate cells comprises isolated CD34⁺ cells not isolated from said perfusate. In a more specific embodiment, said CD34⁺ cells are isolated from placenta. In another more specific embodiment, said CD34⁺ cells are isolated from umbilical cord blood, placental blood, peripheral blood, or bone marrow. In another more specific embodiment, the CD34⁺ cells are CD45⁻. In another more specific embodiment, said CD34⁺ cells, or said CD34^{+CD}45⁻ cells, express a higher level of CD31, CXCR4 or VEGFR than an equivalent number of CD34⁺ cells from umbilical cord blood. In another specific embodiment, said placental perfusate cells are administered on a scaffold or matrix. In another specific embodiment, said placental perfusate cells are administered intravenously.

In certain embodiments of the above methods, the CD34⁺ placental cells are isolated from placental perfusate, e.g., isolated from placental perfusate cells. In certain embodiments, the cells are CD44⁻. In certain embodiments, the cells are CD9⁺, CD54⁺, CD90⁺, or CD166⁺. In certain embodiments, the cells are CD9⁺, CD54⁺, CD90⁺, and CD166⁺. In certain embodiments, the cells are CD31⁺, CD117⁺, CD133⁺, or CD200⁺. In certain embodiments, the cells are CD31⁺, CD117⁺, CD133⁺, and CD200⁺. In certain embodiments, said CD34⁺ cells are CD34^{+CD}45⁻ cells. In certain other embodiments, said CD34⁺ cells express a higher level of CD31, CXCR4 or VEGFR than an equivalent number of CD34⁺ cells from umbilical cord blood.

In certain embodiments, CD34⁺ cells are combined with placental perfusate or placental perfusate cells. In more specific embodiments, the CD34⁺ cells are placental cells. In more specific embodiments, the CD34⁺ cells are placental endothelial progenitor cells. In other specific embodiments, the CD34⁺ cells are hematopoietic cells, e.g., placental CD34⁺ hematopoietic stem cells. In specific embodiments, the ratio of hematopoietic cells to placental perfusate cells is about 100:1, 95:5, 90:10, 85:15, 80:20, 75:25, 70:30, 65:35, 60:40, 55:45:50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like. In certain other embodiments, CD34⁺ cells from a source other than placenta (e.g., from umbilical cord blood, placental blood, peripheral blood, bone marrow, or the like) are combined with CD34⁺ placental cells. In specific embodiments, the ratio of non-placental CD34⁺ cells to CD34⁺ placental cells is about 100:1, 95:5, 90:10, 85:15, 80:20, 75:25, 70:30, 65:35, 60:40, 55:45:50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

The placental perfusate in certain embodiments comprises tissue culture plastic-adherent placental stem cells. The adherent stem cells are the cells described in detail in U.S. Pat. Nos. 7,045,148; 7,255,879; 7,311,904 and 7,311,905; and in U.S. Application Publication Nos. 2007/0275362 and 2008/0032401, the disclosures of which are hereby incorporated by reference in their entireties. The adherent placental stem cells exhibit one or more characteristics of a stem cell (e.g., exhibit markers associated with stem cells, replicate at least 10-20 times in culture in an undifferentiated state, have the ability to differentiate into adult cells representative of the three germ layers, etc.), and can adhere to a tissue culture substrate (e.g., tissue culture plastic such as the surface of a tissue culture dish or multiwell plate).

In one embodiment, the adherent placental stem cells are CD200⁺ or HLA-G⁺. In a specific embodiment, said cell is CD200⁺ and HLA-G⁺. In a specific embodiment, said stem cells are CD73⁺ and CD105⁺. In another specific embodiment, said stem cells are CD34⁻, CD38⁻ or CD45⁻. In another specific embodiment, said stem cells are CD34⁻, CD38⁻ and CD45⁻. In another specific embodiment, said stem cells are CD34⁻, CD38⁻, CD45⁻, CD73⁺ and CD105⁺. In another specific embodiment, said stem cell facilitates the formation of one or more embryoid-like bodies from a population of isolated placental cells comprising placental stem cells when said population is cultured under conditions that allow formation of embryoid-like bodies.

In another embodiment, the adherent placental stem cells are CD73⁺, CD105⁺, and CD200⁺. In a specific embodiment, said stem cells are HLA-G⁺. In another specific embodiment, said stem cells are CD34⁻, CD38⁻ or CD45⁻. In another specific embodiment, said stem cells are CD34⁻, CD38⁻ and CD45⁻. In a more specific embodiment, said stem cells are CD34⁻, CD38⁻, CD45⁻, and HLA-G⁺. In another specific embodiment, said stem cells facilitate development of one or more embryoid-like bodies from a population of isolated placental cells comprising the stem cell when said population is cultured under conditions that allow formation of embryoid-like bodies.

In another embodiment, the adherent placental stem cells are CD200⁺ and OCT-4⁺. In a specific embodiment, the stem cells are CD73⁺ and CD105⁺. In another specific embodiment, said stem cells are HLA-G⁺. In another specific embodiment, said stem cells are CD34⁻, CD38⁻ or CD45⁻. In another specific embodiment, said stem cells are CD34⁻, CD38⁻ and CD45⁻. In a more specific embodiment, said stem cells are CD34⁻, CD38⁻, CD45⁻, CD73⁺, CD105⁺ and HLA-G⁺. In another specific embodiment, said stem cells facilitate the formation of one or more embryoid-like bodies from a population of isolated placental cells comprising placental stem cells when said population is cultured under conditions that allow formation of embryoid-like bodies.

In another embodiment, the adherent placental stem cells are CD73⁺ and CD105⁺ and facilitate the formation of one or more embryoid-like bodies in a population of isolated placental cells comprising said stem cell when said population is cultured under conditions that allow formation of embryoid-like bodies. In a specific embodiment, said stem cells are CD34⁻, CD38⁻ or CD45⁻. In another specific embodiment, said stem cells are CD34⁻, CD38⁻ and CD45⁻. In another specific embodiment, said stem cells are OCT4⁺. In a more specific embodiment, said stem cell is OCT4+, CD34⁻, CD38⁻ and CD45⁻.

In another embodiment, the adherent placental stem cells are CD73⁺, CD105⁺ and HLA-G⁺. In a specific embodiment, said stem cells are CD34⁻, CD38⁻ or CD45⁻. In another specific embodiment, said stem cells are CD34⁻, CD38⁻ and CD45⁻. In another specific embodiment, said stem cells are OCT-4⁺. In another specific embodiment, said stem cells are CD200⁺. In a more specific embodiment, said stem cells are CD34⁻, CD38⁻, CD45⁻, OCT-4⁺ and CD200⁺. In another specific embodiment, said stem cells facilitate the formation of one or more embryoid-like bodies from a population of isolated placental cells comprising placental stem cells in culture under conditions that allow formation of embryoid-like bodies.

In another embodiment, the adherent placental stem cells are OCT-4⁺ and facilitate formation of one or more embryoid-like bodies in a population of isolated placental cells comprising said stem cell when cultured under conditions that allow formation of embryoid-like bodies. In a specific embodiment, said stem cell is CD73⁺ and CD105⁺. In another specific embodiment, said stem cell is CD34⁻, CD38⁻, or CD45⁻. In another specific embodiment, said stem cell is CD200⁺. In a more specific embodiment, said stem cell is CD73⁺, CD105⁺, CD200⁺, CD34⁻, CD38⁻, and CD45⁻.

In another embodiment, the perfusate or perfusate cells comprise an isolated population of the placental stem cells described herein that is produced according to a method comprising perfusing a mammalian placenta that has been drained of cord blood and perfused to remove residual blood; perfusing said placenta with a perfusion solution; and collecting said perfusion solution, wherein said perfusion solution after perfusion comprises a population of placental cells that comprises placental stem cells; and isolating a plurality of said placental stem cells from said population of cells. In a specific embodiment, the perfusion solution is passed through both the umbilical vein and umbilical arteries and collected after it exudes from the placenta. In another specific embodiment, the perfusion solution is passed through the umbilical vein and collected from the umbilical arteries, or passed through the umbilical arteries and collected from the umbilical vein.

In more specific embodiments, the adherent placental stem cells express one or more genes at a detectably higher level than a bone marrow-derived mesenchymal stem cell, wherein said one or more genes are selected from the group consisting of ACTG2, ADARB1, AMIGO2, ATRS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PJP2, RTN1, SERPINB9, ST3GAL6, ST6GALNAC5, SLC12A8, TCF21, TGFB2, VTN, and ZC3H12A, and wherein said bone marrow derived stem cell has undergone a number of passages in culture equivalent to a number of passages for said placental stem cell.

Also provided herein is the use of compositions, e.g., pharmaceutical compositions, that comprise placental perfusate or perfusate cells. In specific embodiments, the placental perfusate or placental perfusate cells are supplemented with a plurality of CD34⁺ placental cells and/or adherent placental stem cells. In a specific embodiment, the composition comprises placental perfusate or placental perfusate cells and one or more agents that induce the formation of vessels or vessel-like structures from said perfusate or perfusate cells. In a more specific embodiment, said agents comprise TGF-β, FGF, plasminogen, tPA, and one or more matrix metalloproteases.

In another specific embodiment, any of the foregoing compositions comprises a matrix. In a more specific embodiment, said matrix is a three-dimensional scaffold. In another more specific embodiment, said matrix comprises collagen, gelatin, laminin, fibronectin, pectin, ornithine, or vitronectin. In another more specific embodiment, the matrix is an amniotic membrane or an amniotic membrane-derived biomaterial. In another more specific embodiment, said matrix comprises an extracellular membrane protein. In another more specific embodiment, said matrix comprises a synthetic compound. In another more specific embodiment, said matrix comprises a bioactive compound. In another more specific embodiment, said bioactive compound is a growth factor, cytokine, antibody, or organic molecule of less than 5,000 daltons. In certain embodiments, the matrix is a synthetic degradable polymer such as, for example, polylactic acid or polyglycolic acid. In certain embodiments, the matrix is an implantable scaffolding substrate. In certain embodiments, the implantable scaffolding substrate is a collagen substrate or a hyaluronic acid substrate. In certain embodiments, the implantable scaffolding substrate is a collagen substrate.

In still another aspect, provided herein is a method for formulating a matrix, comprising combining placental perfusate or perfusate cells with an implantable scaffolding substrate. In certain embodiments, the stem cells are nonadherent. In certain embodiments, the stem cells are CD34⁺. In another aspect, provided herein is a method for formulating an injectable composition, comprising combining placental perfusate or perfusate cells with injectable hyaluronic acid or collagen. In certain embodiments, the stem cells are nonadherent. In certain embodiments, the stem cells are CD34⁺.

In certain embodiments, the placental perfusate cells, or composition, e.g, pharmaceutical composition, comprising the placental perfusate cells, is contained in a container. The container, in one embodiment, is a bag suitable for the intravenous delivery of a liquid. In certain embodiments, the container comprises at least, about, or at most 1×10⁶, 5×10⁶, 1×10⁷, 5×10⁷, 1×10⁸, 5×10⁸, 1×10⁹, 5×10⁹, or 1×10¹⁰ cells, e.g., placental perfusate cells, placental perfusate cells supplemented with a plurality of CD34⁺ placental cells (e.g., CD34⁺ placental endothelial progenitor cells), or placental perfusate cells supplemented with adherent placental stem cells. In certain embodiments, the container comprises said stem cells. In certain embodiments, the cells have been passaged no more than 5 times, 10 times, or 20 times. In certain embodiments, the cells have been expanded within said container. In certain embodiments, the said cells in said container are contained in a 0.9% NaCl solution.

In another aspect, the provided herein is a method for formulating an matrix, comprising combining placental perfusate or perfusate cells comprising stem cells with an implantable scaffolding substrate. In certain other embodiments, provided herein is a method for formulating an injectable composition, comprising combining a population of stem cells with injectable hyaluronic acid or collagen, wherein said stem cells are CD34⁺ placental cells. In certain embodiments, said stem cells are contained within placental perfusate cells. In certain embodiments, the composition comprises injectable hyaluronic acid. In certain embodiments, the composition comprises injectable collagen.

Further provided herein is the use of cryopreserved placental perfusate or perfusate cells in the compositions and methods provided herein.

### 3.1 Definitions

As used herein, the term “SH2” refers to an antibody that binds an epitope on the marker CD105. Thus, cells that are referred to as SH2⁺ are CD105⁺.

As used herein, the terms “SH3” and SH4” refer to antibodies that bind epitopes present on the marker CD73. Thus, cells that are referred to as SH3⁺ and/or SH4⁺ are CD73⁺.

As used herein, the term “isolated stem cell” means a stem cell that is substantially separated from other, non-stem cells of the tissue, e.g., placenta, from which the stem cell is derived. A stem cell is “isolated” if at least about 50%, 60%, 70%, 80%, 90%, 95%, or at least 99% of the non-stem cells with which the stem cell is naturally associated are removed from the stem cell, e.g., during collection and/or culture of the stem cell.

As used herein, the term “population of isolated cells” means a population of cells that is substantially separated from other cells of the tissue, e.g., placenta, from which the population of cells is derived. A stem cell is “isolated” if at least about 50%, 60%, 70%, 80%, 90%, 95%, or at least 99% of the cells with which the population of cells, or cells from which the population of cells is derived, is naturally associated are removed from the stem cell, e.g., during collection and/or culture of the stem cell.

As used herein, “placental perfusate” means perfusion solution that has been passed through at least part of a placenta, e.g., a human placenta, e.g., through the placental vasculature, including a plurality of cells collected by the perfusion solution during passage through the placenta.
As used herein, “placental perfusate cells” means nucleated cells, e.g., total nucleated cells, isolated from, or isolatable from, placental perfusate.

As used herein, the term “placental stem cell” refers to a stem cell or progenitor cell that is derived from a mammalian placenta, regardless of morphology, cell surface markers, or the number of passages after a primary culture. The term “placental stem cell” as used herein does not, however, refer to a trophoblast. A cell is considered a “stem cell” if the cell retains at least one attribute of a stem cell, e.g., a marker or gene expression profile associated with one or more types of stem cells; the ability to replicate at least 10-40 times in culture, the ability to differentiate into cells of all three germ layers; the lack of adult (i.e., differentiated) cell characteristics, or the like. The terms “placental stem cell” and “placenta-derived stem cell” may be used interchangeably.

As used herein, a stem cell is “positive” for a particular marker when that marker is detectable. For example, a placental stem cell is positive for, e.g., CD73 because CD73 is detectable on placental stem cells in an amount detectably greater than background (in comparison to, e.g., an isotype control). A cell is also positive for a marker when that marker can be used to distinguish the cell from at least one other cell type, or can be used to select or isolate the cell when present or expressed by the cell.

As used herein, a “matrix” refers to a three-dimensional substance that is characterized by pores dispersed throughout the substance. The pores are suitable, for example, for growth of cells, e.g., stem cells, PDACs, and/or osteogenic cells, within the matrix. Exemplary matrices include, but are not limited to, a β-tricalcium phosphate substrate, a β-tricalcium phosphate-collagen substrate, a collagen substrate, a calcium phosphate substrate, a mineralized human placental collagen substrate, a hyaluronic acid substrate, and a ceramic substrate. Preferably, the matrix can be mineralized by an osteogenic cell present in the pores of the matrix.

### 4. BRIEF DESCRIPTION OF THE FIGURES

FIG. 1 depicts percentage of nucleated perfusate cells expressing CD34 and/or CD45 in cord blood (CB) or human placental perfusate (HPP).

FIG. 2 depicts percentage of CD34⁺ cells from cord blood (CB) or human placental perfusate (HPP) expressing CD31, CXCR4 and/or VEGFR.

FIG. 3 depicts gene expression analysis in HPP CD34^{+CD}45⁻ and CD34^{+CD}45⁺ cells by qRT-PCR. Relative expression of CD34 and CD45 in human placental perfusate CD34⁺, CD45⁻ and CD34⁺, CD45⁺ cells is normalized to expression of CD34 and CD45 in CD34⁺ cells from umbilical cord blood. Relative quantitation (RQ) (Y axis) is presented as 2^{-ΔΔCt}.

FIG. 4 depicts CFU-Hill colonies stained with Gill's Modified Hematoxylin stain, magnification 200×. Colonies developed from cultures of human placental perfusate cells cultured for 2 weeks in ENDOCULT® medium.

FIG. 5 depicts vessel formation by HPP cells cultured for 18-24 hours on ECMatrix at 10⁶ cells per well of a 96 well plate. Magnification: 200×.

FIG. 6 depicts in vivo bone forming activity by HPP.

FIGS. 7A, 7B: Image taken in the center of scaffold after 21 days post implantation (A) Vitoss and HPP cells (B) Vitoss without cells. As seen in FIG. 6A, a number of CD34 positive cells (arrows) are observable close to vessels. Original magnification 200×, scale bar 100 μm; original colors: aSMA red (AF594), CD34 green (flourescein).

FIGS. 8A, 8B: Image taken in the center of scaffold after 42 days post implantation (A) Vitoss and HPP (B) Vitoss without cells. As seen in FIG. 7A, CD34 positive cells (arrows) are not observable close to vessels. Original magnification 200×, scale bar 100 um, aSMA red (AF594).

FIG. 9: Image analysis showing statistically significant enhancement of angiogenesis in group with HPP cells at 21 days for two animals. Y axis—percent expression of alpha smooth muscle actin.

### 5. DETAILED DESCRIPTION

### 5.1 Methods of Treatment Using Placental Perfusate

Provided herein are methods of producing vasculogenic or angiogenic cells from placental perfusate, and the use of such cells, and the use of placental perfusate or placental perfusate cells, e.g., total nucleated cells from placental perfusate, either alone or in combination with CD34⁺ placental cells (e.g., CD34⁺ placental endothelial progenitor cells) and/or adherent placental stem cells, e.g., the adherent placental stem cells described in Section 5.3, below, in the treatment of individuals having a cardiac or vascular insufficiency, disease, disorder or condition. In a more specific embodiment, said disease, disorder or condition is peripheral vascular disease, acute or chronic myocardial infarct, cardiomyopathy, congestive or chronic heart failure, cardiovascular ischemia, hypertensive pulmonary vascular disease, peripheral arterial disease, or rheumatic heart disease.

In one aspect, provided herein is a method of producing angiogenic or vasculogenic cells, e.g., with the ability to form vasculature, comprising contacting placental perfusate or perfusate cells with conditions in which a plurality of said cells differentiate into cells of the vascular or cardiac system, e.g., into vascular cells, e.g., endothelial cells, or into cardiac cells. In a specific embodiment, said contacting is in vivo. In another specific embodiments, said contacting is in vitro, e.g., culturing said perfusate or said perfusate cells under conditions in which the cells either differentiate into cells of the vascular or cardiac system, or display characteristics of such cells. In a more specific embodiments, said one or more characteristics comprise the formation of vessels or vessel-like structures. In another specific embodiment, said culturing comprises contacting said perfusate cells, e.g., said CD34⁺ placental cells, with transforming growth factor-beta (TGF-β), fibroblast growth factor (FGF), plasminogen, tissue plasminogen activator (tPA) and one or more matrix metalloproteases. In another embodiment, said contacting comprises contacting said perfusate cells with VEGF (50 to 200 ng/mL), TGF-β (1 to 5 ng/mL), FGF (10 to 50 ng/mL) and one or more matrix metalloproteases (1 to 3 Unit/mL each), e.g., wherein said VEGF, TGF-β, FGF and one or more matrix metalloproteases are contained in a matrix, e.g., Matrigel. Said matrix metalloproteases may be any matrix metalloprotease or combinations of matrix metalloproteinases, e.g., a combination of matrix metalloproteinases 1, 3 and 4. In a more specific embodiment, said culturing is for 18-24 hours. In another more specific embodiment, said cells form visible vessel structures after 24 hours of said contacting. In a more specific embodiment, said contacting is under conditions in which said cells produce visible vessel structures after 24 hours, and CD34⁺ cells from umbilical cord blood do not form visible vessel structures, or detectably fewer vessel structures than said perfusate cells or CD34⁺ placental cells. In another specific embodiment, said contacting is performed in vitro. In another specific embodiment, said contacting is performed in vivo. In a more specific embodiments, said in vivo contacting is performed in a mammal. In a more specific embodiment, said mammal is a human.

In certain embodiments, provided herein is a method of forming vessels from a population of placental perfusate cells, comprising contacting said population of cells with conditions that promote the formation of vessels. In a specific embodiment, said population of placental perfusate cells is total nucleated cells from placental perfusate. In another specific embodiment, said contacting is performed in vitro. In another specific embodiment, said contacting is performed in vivo. In another specific embodiment, said population of placental perfusate cells comprises placental perfusate cells isolated from perfusion of a single placenta. In another specific embodiment, said placental perfusate cells are CD34⁺ cells. In a more specific embodiment, said CD34⁺ cells are CD34^{+CD}45⁻ cells. In a more specific embodiment, said CD34⁺ cells or CD34^{+CD}45⁻ cells express a higher level of at least one of CD31, CXCR4 or VEGFR than an equivalent number of CD34⁺ cells from umbilical cord blood. In another specific embodiment, said population of placental perfusate cells comprises isolated CD34⁺ cells not isolated from said perfusate (e.g., isolated from umbilical cord blood, placental blood, peripheral blood, bone marrow, or the like). In a more specific embodiment, said CD34⁺ cells are isolated from placenta. In another more specific embodiment, said CD34⁺ cells are isolated from umbilical cord blood, placental blood, peripheral blood, or bone marrow. In another more specific embodiment, said CD34⁺ cells express a higher level of CD31, CXCR4 or VEGFR than an equivalent number of CD34⁺ cells from umbilical cord blood. In another specific embodiment, said CD34⁺ cells are CD34⁺, CD45⁻ cells.

In another specific embodiment, said placental perfusate or said placental perfusate cells comprise placental stem cells or placental progenitor cells, e.g., CD34⁺ placental cells, for example, CD34⁺ placental endothelial progenitor cells. As used herein, the term “CD34⁺ placental cells” refers to CD34⁺ cells obtained from placenta and not from placental blood or umbilical cord blood. In another specific embodiment, said placental perfusate cells, e.g., said CD34⁺ placental cells produce amounts of one or more angiogenesis-related markers at a higher level than an equivalent number of CD34⁺ cells from umbilical cord blood. In a more specific embodiment, said CD34⁺ cells are CD45⁻. In specific embodiments, said markers comprise CD31, VEGF-R and/or CXCR4. In other embodiments, the CD34⁺ cells are CD44⁻. In certain embodiments, the CD34⁺ cells are CD9⁺, CD54⁺, CD90⁺, or CD166⁺. In certain embodiments, the CD34⁺ cells are CD9⁺, CD54⁺, CD90⁺, and CD166⁺. In certain embodiments, the CD34⁺ cells are CD31⁺, CD117⁺, CD133⁺, or CD200⁺. In certain embodiments, the CD34⁺ cells are CD31⁺, CD117⁺, CD133⁺, and CD200⁺. In certain embodiments, said CD34⁺ cells are CD34^{+CD}45 cells. In certain other embodiments, said CD34⁺ cells express a higher level of CD31, CXCR4 or VEGFR than an equivalent number of CD34⁺ cells from umbilical cord blood.

In certain embodiments, any of the CD34⁺ cells described herein, or populations of CD34⁺ cells, are expanded.

In another aspect, provided herein is a method for treating an individual having a cardiac or vascular insufficiency or defect, e.g., by promoting angiogenesis or angiogenesis in the individual, comprising administering to the individual placental perfusate or placental perfusate cells in an amount sufficient to produce a detectable improvement in, or reduction in the worsening of, one or more symptoms of the cardiac or vascular insufficiency. In a specific embodiment, the placental perfusate or placental perfusate cells are contained within an implantable or injectable composition. In another specific embodiment, the placental perfusate or placental perfusate cells are contained within a composition as provided herein. In another specific embodiment, the placental perfusate or placental perfusate cells are supplemented with a plurality of CD34⁺ placental cells, placental adherent cells, or both.

In another embodiment, provided herein is a method of treating an individual having a cardiac or vascular disease, disorder, condition or insufficiency, comprising administering human placental perfusate cells to said individual in an amount sufficient to treat said disease, disorder, condition or insufficiency. In a specific embodiment, said disease, disorder, condition or insufficiency is peripheral vascular disease, acute or chronic myocardial infarct, cardiomyopathy, congestive or chronic heart failure, cardiovascular ischemia, hypertensive pulmonary vascular disease, peripheral arterial disease, or rheumatic heart disease. In another specific embodiment, said placental perfusate cells are total nucleated cells from placental perfusate. In another specific embodiment, said population of placental perfusate cells comprises placental perfusate cells isolated from perfusion of a single placenta. In another specific embodiment, said population of placental perfusate cells comprises isolated CD34⁺ cells not isolated from said perfusate. In a more specific embodiment, said CD34⁺ cells are isolated from placenta. In another more specific embodiment, said CD34⁺ cells are isolated from umbilical cord blood, placental blood, peripheral blood, or bone marrow. In another more specific embodiment, said CD34⁺ cells express a higher level of CD31, CXCR4 or VEGFR than an equivalent number of CD34⁺ cells from umbilical cord blood. In another specific embodiment, said placental perfusate cells are administered on a scaffold or matrix. In another specific embodiment, said placental perfusate cells are administered intravenously.

The placental perfusate, perfusate cells, combinations of perfusate or perfusate cells with other cells, and compositions, e.g., pharmaceutical compositions, comprising the same, are described in detail in the following sections.

### 5.2 Methods of Obtaining Placental Perfusate and Perfusate Cells

Provided herein are methods of obtaining placental perfusate and placental perfusate cells from a mammalian placenta. In all of the embodiments described herein, the preferred perfusate is human placental perfusate, and the preferred perfusate cells are human placental perfusate cells.

5.2.1 Collection and Handling of Placenta

Generally, a human placenta is recovered shortly after its expulsion after birth. In a preferred embodiment, the placenta is recovered from a patient after informed consent and after a complete medical history of the patient is taken and is associated with the placenta. Preferably, the medical history continues after delivery. Such a medical history can be used to coordinate subsequent use of the placenta or the stem cells harvested therefrom. For example, human placental stem cells can be used, in light of the medical history, for personalized medicine for the infant associated with the placenta, or for parents, siblings or other relatives of the infant.

Prior to recovery of placental stem cells, the umbilical cord blood and placental blood are removed. In certain embodiments, after delivery, the cord blood in the placenta is recovered. The placenta can be subjected to a conventional cord blood recovery process. In one embodiment, the placenta is exsanguinated, e.g., using a needle or cannula with the aid of gravity (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. patent application Ser. No. 11/230,760, filed Sep. 19, 2005. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood. The placenta is preferably stored in an anticoagulant solution at a temperature of 5 to 25° C. (centigrade). Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental stem cells are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

5.2.2 Placental Perfusion

Methods of perfusing mammalian placentae are disclosed, e.g., in Hariri, U.S. Application Publication No. 2002/0123141, and in U.S. application Ser. No. 11/648,812, entitled “Improved Composition for Collecting and Preserving Organs” filed on Dec. 28, 2006.

Perfusate can be obtained by passage of perfusion solution, e.g., saline solution, culture medium or cell collection compositions, as described above, through the placental vasculature. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion solution through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins, that is, is passed through only the placental vasculature (fetal tissue).

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796, the disclosure of which is hereby incorporated by reference herein. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

In one embodiment, the proximal umbilical cord is clamped during perfusion, and more preferably, is clamped within 4-5 cm (centimeter) of the cord's insertion into the placental disc.

In certain embodiments, cord blood is removed from the placenta prior to perfusion (e.g., by gravity drainage), but the placenta is not flushed (e.g., perfused) with solution to remove residual blood. The first collection of perfusion fluid from a mammalian placenta in such an embodiment is generally colored with residual red blood cells of the cord blood and/or placental blood. The perfusion fluid becomes more colorless as perfusion proceeds and the residual cord blood cells are washed out of the placenta. Generally from 30 to 100 mL of perfusion fluid is adequate to initially remove residual cord blood cells.

In other embodiments, cord blood is removed from the placenta prior to perfusion (e.g., by gravity drainage), and the placenta is flushed (e.g., perfused) with solution to remove residual blood, prior to perfusion to recover placental stem cells or placental perfusate cells.

The volume of perfusion liquid used to perfuse the placenta may vary depending upon the number of placental cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with a cell collection composition, or a standard perfusion solution (e.g., a normal saline solution such as phosphate buffered saline (“PBS”) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., total nucleated cells. Perfusates from different time points can also be pooled.

5.2.3 Placental Cell Collection Composition

Perfusate can be collected from the placenta by perfusion of the placenta with any physiologically-acceptable solution, e.g., a saline solution, culture medium, or a more complex cell collection composition. A cell collection composition is described in detail in related U.S. Application Publication No. 2007/0190042, both of which are incorporated herein by reference in their entireties.

The cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

The cell collection composition can comprise one or more tissue-degrading enzymes, e.g., a metalloprotease, a serine protease, a neutral protease, a hyaluronidase, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/l to about 100,000 units/l); or an amiloride containing compound (e.g., amiloride, ethyl isopropyl amiloride, hexamethylene amiloride, dimethyl amiloride or isobutyl amiloride present at about 1.0 μM to about 5 μM).

5.2.4 Placental Perfusate and Placental Perfusate Cells

Placental perfusate comprises a heterogeneous collection of cells. Typically, placental perfusate is depleted of erythrocytes prior to use. Such depletion can be carried out by known methods of separating red blood cells from nucleated blood cells. In certain embodiment, the perfusate or perfusate cells are cryopreserved. In certain other embodiments, the placental perfusate comprises, or the perfusate cells comprise, only fetal cells, or a combination of fetal cells and maternal cells.

Typically, placental perfusate from a single placental perfusion comprises about 100 million to about 500 million nucleated cells. In certain embodiments, the placental perfusate or perfusate cells comprise CD34⁺ cells, e.g., hematopoietic stem or progenitor cells or endothelial progenitor cells. Such cells can, in a more specific embodiment, comprise CD34^{+CD}45⁻ stem or progenitor cells, CD34^{+CD}45⁺ stem or progenitor cells, myeloid progenitors, lymphoid progenitors, and/or erythroid progenitors. In other embodiments, placental perfusate and placental perfusate cells comprise adherent placental stem cells, e.g., CD34 stem cells, e.g., the cells described in Section 5.1, above. In other embodiment, the placental perfusate and placental perfusate cells comprise, e.g., endothelial progenitor cells, osteoprogenitor cells, and natural killer cells. In certain embodiments, placental perfusate as collected from the placenta and depleted of erythrocytes, or perfusate cells isolated from such perfusate, comprise about 6-7% natural killer cells (CD3⁻, CD56⁺); about 21-22% T cells (CD3⁺); about 6-7% B cells (CD19⁺); about 1-2% endothelial progenitor cells (CD34⁺, CD31⁺); about 2-3% neural progenitor cells (nesting); about 2-5% hematopoietic progenitor cells (CD34⁺); and about 0.5-1.5% adherent placental stem cells (e.g., CD34, CD117⁻, CD105⁺ and CD44⁺), as determined, e.g. by flow cytometry, e.g., by FACS analysis.

The CD34⁺ stem or progenitor cells in human placental perfusate express detectably higher levels of angiogenesis-related markers, e.g., CD31, VEGF-R and/or CXCR4 than do an equivalent number of CD34⁺ cells isolated from umbilical cord blood. In certain embodiments, human placental perfusate mononuclear cells from a single perfusion that are cultured in ENDOCULT® medium with VEGF (for growth of CFU-Hill colonies; StemCell Technologies, Inc.) generate up to about 20, e.g., about 1, 2, 3, 4, 5, 6, 7, 8, 9, 10, 11, 12, 13, 14, 15, 16, 17, 18, 19 or 20 CFU-Hill colonies (endothelial cell progenitors). Development of CFU-Hill colonies in liquid culture can be demonstrated and assessed, e.g., by measuring uptake of diacetylated low density lipoprotein (Dil-acLDL) by endothelial progenitor cells obtained from human placental perfusate at, e.g., seven days of culture in ENDOCULT® medium.

In other embodiments, the CD34⁺ cells are CD44⁻. In certain embodiments, the CD34⁺ cells are CD9⁺, CD54⁺, CD90⁺, or CD166⁺. In certain embodiments, the CD34⁺ cells are CD9⁺, CD54⁺, CD90⁺, and CD166⁺. In certain embodiments, the CD34⁺ cells are CD31⁺, CD117⁺, CD133⁺, or CD200⁺. In certain embodiments, the CD34⁺ cells are CD31⁺, CD117⁺, CD133⁺, and CD200⁺.

In one embodiment, the human placental perfusate cells produce vessels or vessel-like structures when cultured. Vessel formation of HPP cells can be demonstrated, e.g., by culture of the cells, e.g., about 5×10⁵ cells on a matrix, e.g., ECMATRIX™, in the presence of TGF-β (transforming growth factor beta), fibroblast growth factor (FGF), plasminogen, tissue plasminogen activator (tPA), and matrix metalloproteinases (MMPs). Vessels and vessel-like structures form after about 18-24 hours. No significant vessel formation is seen in cord blood mononuclear cells cultured under the same conditions. Vessel formation can also be seen by culturing perfusate cells in contact with VEGF (50 to 200 ng/mL), TGF-β (1 to 5 ng/mL), FGF (10 to 50 ng/mL) and one or more matrix metalloproteases (1 to 3 Unit/mL each), e.g., wherein said VEGF, TGF-β, FGF and one or more matrix metalloproteases are contained in a matrix, e.g., Matrigel.

Moreover, CD34^{+CD}45⁻ cells from human placental perfusate have a detectably higher expression of angiogenesis related markers CD31 and/or VEGFR than CD34^{+CD}45⁺ cells.

Typically, placental perfusate and perfusate cells have low expression of MHC class I compared to umbilical cord blood cells, and are largely negative for MHC class II markers.

5.2.5 Isolation, Sorting, and Characterization of Placental Cells

Cells from mammalian placenta, e.g., perfusate cells or stem cells from perfusate, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh cell collection composition as described above, or a medium suitable for stem cell maintenance, e.g., IMDM serum-free medium containing 2 U/ml heparin and 2 mM EDTA (GibcoBRL, NY). The total mononuclear cell fraction can be isolated, e.g., using Lymphoprep (Nycomed Pharma, Oslo, Norway) according to the manufacturer's recommended procedure.

As used herein, “isolating” placental cells, e.g., stem or progenitor cells from placental perfusate or placental perfusate cells, means to remove at least about 20%, 30%, 40%, 50%, 60%, 70%, 80%, 90%, 95% or 99% of the cells with which the cells are normally associated in the intact mammalian placenta. A cell from an organ is “isolated” when it is present in a population of cells that comprises fewer than 50% of the cells with which the cell is normally associated in the intact organ.

Placental cells, e.g., the adherent placental stem cells described above, obtained by perfusion can, for example, be further, or initially, isolated by differential trypsinization using, e.g., a solution of 0.05% trypsin with 0.2% EDTA (Sigma, St. Louis Mo.). Differential trypsinization of adherent placental stem cells is possible because the stem cells typically detach from plastic surfaces within about five minutes whereas other adherent cell populations in placental perfusate typically require more than 20-30 minutes incubation. The detached placental stem cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺ Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, OCT-4 and/or HLA-G. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34⁻ cells are retained, and cells that are CD200⁺ HLA-G⁺, are separated from all other CD34⁻ cells. In another embodiment, cells from placenta are based on their expression of markers CD200 and/or HLA-G; for example, cells displaying either of these markers are isolated for further use. Cells that express, e.g., CD200 and/or HLA-G can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental cells are sorted by expression, or lack thereof, of CD200, HLA-G, CD73, CD105, CD34, CD38 and CD45, and placental cells that are CD200⁺, HLA-G⁺, CD73⁺, CD105⁺, CD34⁻, CD38⁻ and CD45⁻ are isolated from other placental cells for further use.

In another embodiment, placental perfusate cells are sorted based on their expression of CD34⁺ and expression of one or more angiogenic markers, e.g., CXCR4, VEGFR and/or CD31.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Placental cells can also be characterized and/or sorted based on cell morphology and growth characteristics. For example, placental cells, e.g., adherent placental stem cells, can be characterized as having, and/or selected on the basis of, e.g., a fibroblastoid appearance in culture. Placental cells can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4⁺ placental cells that produce one or more embryoid-like bodies in culture are isolated from other placental cells.

In another embodiment, placental cells, e.g., placental perfusate or perfusate cells can be identified and characterized by a colony forming unit assay. Colony forming unit assays are commonly known in the art.

Placental perfusate or perfusate cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Placental stem cells can be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.3 Adherent Placental Stem Cells

Adherent placental stem cells are stem cells, obtainable from a placenta or part thereof, that adhere to a tissue culture substrate and have the capacity to differentiate into non-placental cell types. Adherent placental stem cells can be either fetal or maternal in origin (that is, can have the genotype of either the mother or fetus). Populations of adherent placental stem cells, or populations of cells comprising placental stem cells, can comprise placental stem cells that are solely fetal or maternal in origin, or can comprise a mixed population of placental stem cells of both fetal and maternal origin. The placental stem cells, and populations of cells comprising the placental stem cells, can be identified and selected by the morphological, marker, and culture characteristic discussed below. The adherent placental stem cells usable in the compositions and methods described herein, and methods of obtaining and culturing such cells, are described in detail in U.S. Pat. Nos. 7,045,148; 7,255,879; 7,311,904 and 7,311,905; and in U.S. Application Publication Nos. 2007/0275362 and 2008/0032401, the disclosures of which are hereby incorporated by reference in their entireties.

5.3.1 Physical and Morphological Characteristics

The adherent placental stem cells usable in the compositions and methods provided herein, when cultured in primary cultures or in cell culture, adhere to the tissue culture substrate, e.g., tissue culture container surface (e.g., tissue culture plastic). Adherent placental stem cells in culture assume a generally fibroblastoid, stellate appearance, with a number of cyotplasmic processes extending from the central cell body. The adherent placental stem cells are, however, morphologically distinguishable from fibroblasts cultured under the same conditions, as the adherent placental stem cells exhibit a greater number of such processes than do fibroblasts. Morphologically, adherent placental stem cells are also differentiable from hematopoietic stem cells, which generally assume a more rounded, or cobblestone, morphology in culture.

5.3.2 Cell Surface, Molecular and Genetic Markers

Isolated adherent placental stem cells, and populations of adherent placental stem cells, express a plurality of markers that can be used to identify and/or isolate the stem cells, or populations of cells that comprise the stem cells. Adherent placental stem cells, and stem cell populations (that is, two or more placental stem cells) include stem cells and stem cell-containing cell populations obtained directly from the placenta, or any part thereof (e.g., amnion, chorion, placental cotyledons, umbilical cord, and the like).

Adherent placental stem cells generally express the markers CD73, CD105, CD200, HLA-G, and/or OCT-4, and do not express CD34, CD38, or CD45. Placental stem cells can also express HLA-ABC (MHC-1) and HLA-DR.

In one embodiment, isolated adherent placental stem cells are CD200⁺ or HLA-G⁺. In a specific embodiment, the stem cell is CD200⁺ and HLA-G⁺. In a specific embodiment, said stem cell is CD73⁺ and CD105⁺. In another specific embodiment, said stem cells are CD34⁻, CD38⁻ or CD45⁻. In another specific embodiment, said stem cells are CD34⁻, CD38⁻ and CD45⁻. In another specific embodiment, said stem cells are CD34⁻, CD38⁻, CD45⁻, CD73⁺ and CD105⁺. In another specific embodiment, said CD200⁺ or HLA-G⁺ stem cells facilitate the formation of embryoid-like bodies in a population of placental cells comprising the stem cells, under conditions that allow the formation of embryoid-like bodies.

In another embodiment, isolated adherent placental stem cells are CD73⁺, CD105⁺, and CD200⁺. In another specific embodiment, said stem cells are HLA-G⁺. In another specific embodiment, said stem cells are CD34⁻, CD38⁻ or CD45⁻. In another specific embodiment, said stem cells are CD34⁻, CD38⁻ and CD45⁻. In a more specific embodiment, said stem cells are CD34⁻, CD38⁻, CD45⁻, and HLA-G⁺. In another specific embodiment, said stem cells are CD73⁺, CD105⁺, and CD200⁺ and facilitate the formation of one or more embryoid-like bodies in a population of placental cells comprising the stem cells, when the population is cultured under conditions that allow the formation of embryoid-like bodies.

In another embodiment, isolated adherent placental stem cells are CD200⁺ and OCT-4⁺. In a specific embodiment, the stem cells are CD73⁺ and CD105⁺. In another specific embodiment, said stem cells are HLA-G⁺. In another specific embodiment, said stem cells are CD34⁻, CD38⁻ or CD45⁻. In another specific embodiment, said stem cells are CD34⁻, CD38⁻ and CD45⁻. In a more specific embodiment, said stem cells are CD34⁻, CD38⁻, CD45⁻, CD73⁺, CD105⁺ and HLA-G⁺. In another specific embodiment, the stem cells facilitate the production of one or more embryoid-like bodies by a population of placental cells that comprises the stem cells, when the population is cultured under conditions that allow the formation of embryoid-like bodies.

In another embodiment, isolated adherent placental stem cells are CD73⁺, CD105⁺ and HLA-G⁺. In another specific embodiment, said stem cells are CD34⁻, CD38⁻ or CD45⁻. In another specific embodiment, said stem cells are CD34⁻, CD38⁻ and CD45⁻. In another specific embodiment, said stem cells are OCT-4⁺. In another specific embodiment, said stem cells are CD200⁺. In a more specific embodiment, said stem cells are CD34⁻, CD38⁻, CD45⁻, OCT-4⁺ and CD200⁺. In another specific embodiment, said stem cells facilitate the formation of embryoid-like bodies in a population of placental cells comprising said stem cells, when the population is cultured under conditions that allow the formation of embryoid-like bodies.

In another embodiment, isolated adherent placental stem cells are CD73⁺ and CD105⁺ and facilitates the formation of one or more embryoid-like bodies in a population of isolated placental cells comprising said stem cells when said population is cultured under conditions that allow formation of embryoid-like bodies. In a specific embodiment, said stem cells are CD34⁻, CD38⁻ or CD45⁻. In another specific embodiment, said stem cells are CD34⁻, CD38⁻ and CD45⁻. In another specific embodiment, said stem cells are OCT4⁺. In a more specific embodiment, said stem cells are OCT4⁺, CD34⁻, CD38⁻ and CD45⁻.

In another embodiment, isolated adherent placental stem cells are OCT-4⁺ and facilitate formation of one or more embryoid-like bodies in a population of isolated placental cells comprising said stem cells when cultured under conditions that allow formation of embryoid-like bodies. In a specific embodiment, said stem cells are CD73⁺ and CD105⁺. In another specific embodiment, said stem cells are CD34⁻, CD38⁻, or CD45⁻. In another specific embodiment, said stem cells are CD200⁺. In a more specific embodiment, said stem cells are CD73⁺, CD105⁺, CD200⁺, CD34⁻, CD38⁻, and CD45⁻.

Adherent placental stem cells can be obtained by enzymatic digestion or perfusion, e.g., by perfusion of a mammalian placenta as described above. In a specific embodiment, the perfusion solution is passed through the umbilical vein and collected from the umbilical arteries, or passed through the umbilical arteries and collected from the umbilical vein. Adherent placental stem cells can be substantially exclusively fetal in origin; that is, e.g., greater than 90%, 95%, 99%, or 99.5% of the placental stem cells in the population are fetal in origin. Enzymatic digestion of placental tissue to obtain adherent placental stem cells is described in U.S. Patent Application Publication No. 2007/0275362, the disclosure of which is hereby incorporated herein by reference in its entirety.

Adherent placental stem cells, in other embodiments, express one or more genes at a detectably higher level than comparison to bone marrow-derived mesenchymal stem cells, wherein the one or more gene is/are ACTG2, ADARB1, AMIGO2, ATRS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRC5B, ICAM1, IER3, IGFBP7, IL1A, IL6, IL18, KRT18, KRT8, LIPG, LRAP, MATN2, MEST, NFE2L3, NUAK1, PCDH7, PDLIM3, PJP2, RTN1, SERPINB9, ST3GAL6, ST6GALNAC5, SLC12A8, TCF21, TGFB2, VTN, ZC3H12A, or a combination of any of the foregoing, wherein the cells are grown under equivalent conditions. In a specific embodiment, the placental stem cell-specific or umbilical cord stem cell-specific gene is CD200.

### 5.4 Culture of Placental Perfusate Cells

5.4.1 Culture Media

Isolated placental cells, e.g., perfusate cells, or cells obtained therefrom, e.g., placental stem cells, or placental stem cell population, or cells or placental tissue from which placental stem cells grow out, can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, and extracellular membrane protein (e.g., MATRIGEL (BD Discovery Labware, Bedford, Mass.)).

Placental cells can be cultured in any medium, and under any conditions, recognized in the art as acceptable for the culture of cells, e.g., stem cells. Preferably, the culture medium comprises serum. Placental perfusate cells, or placental stem cells, can be cultured in, for example, DMEM-LG (Dulbecco's Modified Essential Medium, low glucose)/MCDB 201 (chick fibroblast basal medium) containing ITS (insulin-transferrin-selenium), LA+BSA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; α-MEM (minimal essential medium) comprising 10% FBS, GLUTAMAX™ and gentamicin; DMEM comprising 10% FBS, GLUTAMAX™ and gentamicin, etc. A preferred medium is DMEM-LG/MCDB-201 comprising 2% FBS, ITS, LA+BSA, dextrose, L-ascorbic acid, PDGF, EGF, and penicillin/streptomycin.

Other media in that can be used to culture placental cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMEM/12, RPMI 1640, advanced DMEM (Gibco), DMEM/MCDB201 (Sigma), and CELL-GRO FREE.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

Placental perfusate or perfusate cells can be cultured in standard tissue culture conditions, e.g., in tissue culture dishes or multiwell plates. Placental perfusate or perfusate cells can also be cultured using a hanging drop method. In this method, placental stem cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the stem cells are cultured.

5.4.2 Expansion and Proliferation of Placental Cells

Isolated placental cells, e.g., perfusate or perfusate cells or stem cells, or isolated population of such cells (e.g., a stem cell or population of stem cells separated from at least about 50% of the placental cells with which the stem cell or population of stem cells is normally associated in vivo) can be proliferated and expanded in vitro. For example, a population of placental cells can be cultured in tissue culture containers, e.g., dishes, flasks, multiwell plates, or the like, for a sufficient time for the cells to proliferate to 70-90% confluence, that is, until the cells and their progeny occupy 70-90% of the culturing surface area of the tissue culture container.

Placental stem cells can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured at about 0 to about 5 percent by volume CO₂ in air. In some preferred embodiments, the cells are cultured at about 2 to about 25 percent O₂ in air, preferably about 5 to about 20 percent O₂ in air. The cells preferably are cultured at about 25° C. to about 40° C., preferably 37° C. The cells are preferably cultured in an incubator. The culture medium can be static or agitated, for example, using a bioreactor. Placental stem cells preferably are grown under low oxidative stress (e.g., with addition of glutathione, ascorbic acid, catalase, tocopherol, N-acetylcysteine, or the like).

Once 70%-90% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 stem cells, preferably about 50,000 stem cells, are passaged to a new culture container containing fresh culture medium. Typically, the new medium is the same type of medium from which the stem cells were removed. Adherent placental stem cells useful in the methods and compositions provided herein can have been passaged at least 1, 2, 3, 4, 5, 6, 7, 8, 9, 10, 12, 14, 16, 18, or 20 times, or more.

5.4.3 Placental Cell Populations

Placental perfusate, or placental perfusate cells, can be supplemented with adherent placental stem cells, CD34⁺ placental cells (e.g., CD34⁺ placental endothelial progenitor cells, CD34⁺ cells from a source other than placenta (e.g., such as umbilical cord blood, placental blood, peripheral blood, bone marrow, or the like), placental cells that are not stem cells, or cells that are not placental cells.

Isolated placental cell populations, e.g., perfusate or placental perfusate cells, can be combined with one or more populations of non-stem cells or non-placental cells. For example, an isolated population of placental cells can be combined with blood (e.g., placental blood or umbilical cord blood), blood-derived stem cells (e.g., stem cells derived from placental blood or umbilical cord blood), populations of blood-derived nucleated cells, bone marrow-derived mesenchymal cells, bone-derived stem cell populations, crude bone marrow, adult (somatic) stem cells, populations of stem cells contained within tissue, cultured stem cells, populations of fully-differentiated cells (e.g., chondrocytes, fibroblasts, amniotic cells, osteoblasts, muscle cells, cardiac cells, etc.) and the like. Cells in an isolated placental cell population can be combined with a plurality of cells of another type in ratios of about 100,000, 000:1, 50,000, 000:1, 20,000, 000:1, 10,000, 000:1, 5,000, 000:1, 2,000, 000:1, 1,000,000:1, 500,000:1, 200,000:1, 100, 000:1, 50, 000:1, 20, 000:1, 10, 000:1, 5,000:1, 2, 000:1, 1, 000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population. Cells in an isolated placental cell population can be combined with a plurality of cells of a plurality of cell types, as well.

In one, an isolated population of placental perfusate or perfusate cells is combined with a plurality of CD34⁺ cells. Such CD34⁺ cells can be, for example, contained within unprocessed placental, umbilical cord blood or peripheral blood; in total nucleated cells from placental blood, umbilical cord blood or peripheral blood; in an isolated population of CD34⁺ cells from placental blood, umbilical cord blood or peripheral blood; in unprocessed bone marrow; in total nucleated cells from bone marrow; in an isolated population of CD34⁺ cells from bone marrow, or the like. In a specific embodiment, the hematopoietic stem cells are CD34⁺ placental endothelial progenitor cells.

### 5.5 Production of a Placental Cell Bank

Placental perfusate, and placental perfusate cells, can be stored in cell banks. In preferred embodiments, the placental perfusate or perfusate cells are human perfusate or perfusate cells. The perfusate or perfusate cells can be stored in units, e.g., the total perfusate or cells collected from a single placenta, or a single perfusion of a single placenta. Perfusate, or perfusate cells, from a plurality of perfusions, or a plurality of placentae, can be combined into units.

Cells, e.g., stem cells, placental perfusate cells, or combinations thereof, from postpartum placentas can be cultured in a number of different ways to produce a set of lots, e.g., a set of individually-administrable doses, of placental stem cells. Such lots can, for example, be obtained from stem cells from placental perfusate or from enzyme-digested placental tissue. Sets of lots of placental cells, obtained from a plurality of placentas, can be arranged in a bank of placental cells for, e.g., long-term storage. Generally, adherent stem cells are obtained from an initial culture of placental material to form a seed culture, which is expanded under controlled conditions to form populations of cells from approximately equivalent numbers of doublings. Lots are preferably derived from the tissue of a single placenta, but can be derived from the tissue of a plurality of placentas.

In one embodiment, placental cell lots are obtained as follows. Placental perfusate cells are obtained by perfusion of one or more placentas, preferably only through the placental vasculature, preferably from a placenta that has been drained of cord blood and perfused to remove residual blood, the cells in the resulting perfusate are collected by centrifugation, and erythrocytes are removed. These cells are collected and resuspended in a convenient volume of culture medium, and defined as early passage cells.

Early passage cells are then used to seed expansion cultures. Expansion cultures can be any arrangement of separate cell culture apparatuses, e.g., a Cell Factory by NUNC™. Cells in the early passage culture can be subdivided to any degree so as to seed expansion cultures with, e.g., 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴ or 10×10⁴ stem cells. Preferably, from about 2×10⁴ to about 3×10⁴ Passage 0 cells are used to seed each expansion culture. The number of expansion cultures can depend upon the number of early passage cells, and may be greater or fewer in number depending upon the particular placenta(s) from which the stem cells are obtained.

Expansion cultures are grown until the density of cells in culture reaches a certain value, e.g., about 1×10⁵ cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 15, 16, 17, 18, 19 or 20 times prior to use. A record of the cumulative number of population doublings is preferably maintained during expansion culture(s). The cells from early passage culture can be expanded for 2, 3, 4, 5, 6, 7, 8, 9, 10, 12, 14, 16, 18, 20, 22, 24, 26, 28, 30, 32, 34, 36, 38 or 40 doublings, or up to 60 doublings. Preferably, however, the number of population doublings, prior to dividing the population of cells into individual doses, is between about 15 and about 30, preferably about 20 doublings. The cells can be culture continuously throughout the expansion process, or can be frozen at one or more points during expansion.

Cells to be used for individual doses can be frozen, e.g., cryopreserved for later use. Individual doses can comprise, e.g., about 1 million to about 100 million cells per ml, and can comprise between about 10⁶ and about 10⁹ cells in total.

In one embodiment, therefore, a placental stem cell bank can be made by a method comprising: expanding primary culture placental stem cells from a human post-partum placenta for a first plurality of population doublings; cryopreserving said placental stem cells to form a Master Cell Bank; expanding a plurality of placental stem cells from the Master Cell Bank for a second plurality of population doublings; cryopreserving said placental stem cells to form a Working Cell Bank; expanding a plurality of placental stem cells from the Working Cell Bank for a third plurality of population doublings; and cryopreserving said placental stem cells in individual doses, wherein said individual doses collectively compose a placental stem cell bank. In one specific embodiment, said individual doses comprise from about 10⁴ to about 10⁵ placental stem cells. In another specific embodiment, said individual doses comprise from about 10⁵ to about 10⁶ placental stem cells. In another specific embodiment, said individual doses comprise from about 10⁶ to about 10⁷ placental stem cells. In another specific embodiment, said individual doses comprise from about 10⁷ to about 10⁸ placental stem cells. In another specific embodiment, said individual doses comprise from about 10⁸ to about 10⁹ placental stem cells. In another specific embodiment, said individual doses comprise from about 10⁹ to about 10¹⁰ placental stem cells.

In a preferred embodiment, the donor from which the placenta is obtained (e.g., the mother) is tested for at least one pathogen. If the mother tests positive for a tested pathogen, the entire lot from the placenta is discarded. Such testing can be performed at any time during production of placental stem cell lots, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

### 5.6 Preservation of Placental Cells

Placental perfusate, placental perfusate cells, and combinations of placental perfusate or placental perfusate cells with adherent placental stem cells and/or CD34⁺ placental cells, can be preserved, that is, placed under conditions that allow for long-term storage, or conditions that inhibit cell death by, e.g., apoptosis or necrosis.

Cells can be preserved using, e.g., a composition comprising an apoptosis inhibitor, necrosis inhibitor and/or an oxygen-carrying perfluorocarbon, as described in related U.S. Application Publication No. 2007/0190042, entitled “Improved Medium for Collecting Placental Stem Cells and Preserving Organs,” the disclosure of which is hereby incorporated by reference in its entirety.

In one embodiment, a population of placental cells can be preserved by contacting said population of cells with a cell collection composition comprising an inhibitor of apoptosis and an oxygen-carrying perfluorocarbon, e.g., in an emulsion or in separate phases, wherein said inhibitor of apoptosis is present in an amount and for a time sufficient to reduce or prevent apoptosis in the population of stem cells, as compared to a population of cells not contacted with the inhibitor of apoptosis. In various embodiments, said inhibitor of apoptosis is a caspase inhibitor or JNK inhibitor. In another embodiment, the cell collection composition additionally comprises an emulsifier, e.g., lecithin. In another embodiment, said apoptosis inhibitor and said perfluorocarbon are between about 0° C. and about 25° C. at the time of contacting the cells. In another more specific embodiment, said apoptosis inhibitor and said perfluorocarbon are between about 2° C. and 10° C., or between about 2° C. and about 5° C., at the time of contacting the cells. In another more specific embodiment, said contacting is performed during transport of said population of cells. In another more specific embodiment, said contacting is performed during freezing and thawing of said population of cells. The inhibitor of apoptosis can be combined with an organ-preserving compound, such as hydroxyethyl starch, lactobionic acid, raffinose, UW solution (described in U.S. Pat. No. 4,798,824; also known as ViaSpan; see also Southard et al., Transplantation 49(2):251-257 (1990)) or a solution described in Stern et al., U.S. Pat. No. 5,552,267, the disclosures of which are hereby incorporated herein by reference, or a combination thereof.

In another embodiment of the method, placental cells are contacted with a cell collection composition comprising an apoptosis inhibitor and oxygen-carrying perfluorocarbon, organ-preserving compound, or combination thereof, during perfusion. In another embodiment, said cells are contacted during a process of tissue disruption, e.g., enzymatic digestion. In another embodiment, placental cells are contacted with said cell collection compound after collection by perfusion, or after collection by tissue disruption, e.g., enzymatic digestion.

Typically, during placental cell collection, enrichment and isolation, it is preferable to minimize or eliminate cell stress due to hypoxia and mechanical stress. In another embodiment of the method, therefore, placental perfusate, placental perfusate cells, a placental stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of cells is exposed to said hypoxic condition for less than two hours during said preservation. In another more specific embodiment, said population of cells is exposed to said hypoxic condition for less than one hour, or less than thirty minutes, or is not exposed to a hypoxic condition, during collection, enrichment or isolation. In another specific embodiment, said population of cells is not exposed to shear stress during collection, enrichment or isolation.

Placental perfusate and perfusate cells can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 10% (v/v). Cryopreservation medium may comprise additional agents, for example, methylcellulose and/or glycerol. Placental cells are preferably cooled at about 1° C./min during cryopreservation. A preferred cryopreservation temperature is about −80° C. to about −180° C., preferably about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells preferably are thawed at a temperature of about 25° C. to about 40° C., preferably to a temperature of about 37° C.

### 5.7 Uses of Placental Cells

5.7.1 Placental Cell Populations

Provided herein are methods of treating an individual having a cardiac or vascular disease, disorder or insufficiency comprising administering to said individual placental cell populations, including placental perfusate, placental perfusate cells, e.g., total nucleated cells from placental perfusate, and combinations of such with other cells, e.g., endothelial progenitor cells, hematopoietic stem cells or cord blood. As used herein, “treat” encompasses the cure of, remediation of, improvement of, lessening of the severity of, or reduction in the time course of, a cardiac or vascular disease, disorder, condition or insufficiency, or any parameter or symptom thereof. In various embodiments, said disease, disorder, condition or insufficiency is peripheral vascular disease, acute or chronic myocardial infarct, cardiomyopathy, congestive or chronic heart failure, cardiovascular ischemia, hypertensive pulmonary vascular disease, peripheral arterial disease, or rheumatic heart disease.

Placental perfusate cells, and populations of placental perfusate cells, or stem cells obtained therefrom, can be induced to differentiate into a particular cell type, either ex vivo or in vivo, in preparation for administration to an individual in need of stem cells, or cells differentiated from stem cells. For example, placental perfusate or placental perfusate cells can be injected into a damaged organ, and for organ neogenesis and repair of injury in vivo. Such injury can be caused, e.g., by arterial or venous blockage, infarct, ischemia, or the like.

Placental perfusate and perfusate cells can be administered without being cultured under conditions that cause the stem cells to differentiate. Alternately, the perfusate or perfusate cells can be cultured in, e.g., e.g., angiogenic or vasculogenic medium for, e.g., about 1-20 days, prior to administration. In certain embodiments, placental perfusate or perfusate cells can be isolated and seeded on a matrix, then cultured in an angiogenic or vasulogenic medium for, e.g., about 1-20 days. In another embodiment, placental perfusate or perfusate cells can be cultured in, e.g., angiogenic or vasulogenic medium for, e.g., about 1-20 days, then seeded onto a matrix, then cultured in osteogenic medium as described herein for, e.g., about 1-20 days.

Placental perfusate or perfusate cells, alone or in combination with stem cell or progenitor cell populations, e.g., placental stem cells, can be used in the manufacture of a tissue or organ in vitro or in vivo. Cells obtained from the placenta, e.g., perfusate, perfusate cells, placental stem cells or progenitor cells, can be used to seed a matrix, followed by culturing under conditions that cause, or allow, the cells to differentiate and populate the matrix. The tissues and organs obtained by the methods provided herein can be used for a variety of purposes, including research and therapeutic purposes.

In another embodiment, placental perfusate or placental perfusate cells are used for autologous and allogenic transplants, including matched and mismatched HLA type hematopoietic transplants. In one embodiment of the use of placental perfusate and/or perfusate cells as allogenic hematopoietic transplants, the host is treated to reduce immunological rejection of the donor cells, or to create immunotolerance (see, e.g., U.S. Pat. Nos. 5,800,539 and 5,806,529). In another embodiment, the host is not treated to reduce immunological rejection or to create immunotolerance.

Placental perfusate or perfusate cells, either alone or in combination with one or more other stem cell populations, can be used in therapeutic transplantation protocols, e.g., to augment or replace stem or progenitor cells of the liver, pancreas, kidney, lung, nervous system, muscular system, bone, bone marrow, thymus, spleen, mucosal tissue, gonads, or hair. Additionally, placental perfusate or perfusate cells may be used instead of specific classes of progenitor cells (e.g., chondrocytes, hepatocytes, hematopoietic cells, pancreatic parenchymal cells, neuroblasts, muscle progenitor cells, etc.) in therapeutic or research protocols in which progenitor cells would typically be used.

Placental perfusate or perfusate cells can be used to repair damage to tissues and organs resulting from, e.g., trauma, metabolic disorders, or disease. In such an embodiment, a patient can be administered placental perfusate or perfusate cells, alone or combined with other stem or progenitor cell populations, to regenerate or restore tissues or organs which have been damaged as a consequence of disease.

5.7.2 Compositions Comprising Placental Perfusate or Perfusate Cells

Provided herein are compositions comprising, or derived from, placental perfusate or perfusate cells, or biomolecules therefrom. Placental perfusate or perfusate cells can be combined with any physiologically-acceptable or medically-acceptable compound, composition or device for use in, e.g., research or therapeutics.

5.7.2.1 Cryopreserved Placental Perfusate or Perfusate Cells

The placental perfusate or perfusate cells described herein can be preserved, for example, cryopreserved for later use. Methods for cryopreservation of cells, such as stem cells, are well known in the art. Placental stem cell populations can be prepared in a form that is easily administrable to an individual. For example, provided herein is a placental stem cell population that is contained within a container that is suitable for medical use. Such a container can be, for example, a sterile plastic bag, flask, jar, or other container from which the placental stem cell population can be easily dispensed. For example, the container can be a blood bag or other plastic, medically-acceptable bag suitable for the intravenous administration of a liquid to a recipient. The container is preferably one that allows for cryopreservation of the combined stem cell population.

The cryopreserved placental perfusate or placental perfusate cells can comprise placental perfusate or placental perfusate cells derived from a single donor, or from multiple donors. The placental perfusate or placental perfusate cells can be completely HLA-matched to an intended recipient, or partially or completely HLA-mismatched.

Thus, in one embodiment, provided herein is a composition comprising placental perfusate or placental perfusate cells in a container. In a specific embodiment, the placental perfusate or placental perfusate cells are cryopreserved. In another specific embodiment, the container is a bag, flask, or jar. In more specific embodiment, said bag is a sterile plastic bag. In a more specific embodiment, said bag is suitable for, allows or facilitates intravenous administration of said placental stem cell population. The bag can comprise multiple lumens or compartments that are interconnected to allow mixing of the placental stem cells and one or more other solutions, e.g., a drug, prior to, or during, administration. In another specific embodiment, the composition comprises one or more compounds that facilitate cryopreservation of the placental perfusate or placental perfusate cells. In another specific embodiment, said placental perfusate or placental perfusate cells are contained within a physiologically-acceptable aqueous solution. In a more specific embodiment, said physiologically-acceptable aqueous solution is a 0.9% NaCl solution. In another specific embodiment, said placental perfusate or placental perfusate cells comprise placental cells that are HLA-matched to a recipient of said placental perfusate or placental perfusate cells. In another specific embodiment, said placental perfusate or placental perfusate cells comprise placental cells that are at least partially HLA-mismatched to a recipient of said placental perfusate or placental perfusate cells. In another specific embodiment, said placental perfusate or placental perfusate cells are derived from a plurality of donors.

5.7.2.2 Pharmaceutical Compositions

Placental perfusate or placental perfusate cells can be formulated into pharmaceutical compositions for use in vivo. Such pharmaceutical compositions comprise placental perfusate or placental perfusate cells in a pharmaceutically-acceptable carrier, e.g., a saline solution or other accepted physiologically-acceptable solution for in vivo administration. Pharmaceutical compositions provided herein can comprise any of the placental perfusate or placental perfusate cell embodiments. The pharmaceutical compositions can comprise fetal, maternal, or both fetal and maternal placental cells. The pharmaceutical compositions provided herein can further comprise placental cells obtained from a single individual or placenta, or from a plurality of individuals or placentae.

The pharmaceutical compositions provided herein can comprise any number of placental cells. For example, a single unit dose of placental cells, e.g., perfusate cells, can comprise, in various embodiments, about, at least, or no more than 1×10⁵, 5×10⁵, ×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more placental cells.

The cells can be administered, e.g., in a physiologically-acceptable solution, e.g., a saline solution, for example, in phosphate buffered saline, 0.9% NaCl solution, or the like.

The pharmaceutical compositions provided herein can comprise populations of cells, e.g., placental perfusate cells, that comprise 50% viable cells or more (that is, at least about 50% of the cells in the population are functional or living). Preferably, at least about 60% of the cells in the population are viable. More preferably, at least about 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, and the like.

The populations of cells provided herein can be implanted surgically, injected, delivered (e.g., by way of a catheter or syringe), or otherwise administered directly or indirectly to an individual, e.g., at the site in need of repair or augmentation. The populations of cells provided herein, or compositions, e.g., pharmaceutical compositions, can be administered, orally, nasally, intraarterially, parenterally, intravenously, ophthalmically, intramuscularly, subcutaneously, intraperitoneally, intracerebrally, intraventricularly, intracerebroventricularly, intrathecally, intracisternally, intraspinally and/or peri-spinally.

5.7.2.3 Placental Cell Conditioned Media

The placental perfusate, placental perfusate cells, CD34⁺ placental cells, or combinations thereof, e.g., with adherent placental stem cells, can be used to produce conditioned medium, that is, medium comprising one or more biomolecules secreted or excreted by the perfusate or cells. In various embodiments, the conditioned medium comprises medium in which placental cells have grown for at least 1, 2, 3, 4, 5, 6, 7, 8, 9, 10, 11, 12, 13, 14 or more days. In other embodiments, the conditioned medium comprises medium in which placental cells have grown to at least about 30%, 40%, 50%, 60%, 70%, 80%, 90% confluence, or up to 100% confluence. Such conditioned medium can be used to support the culture of a separate population of placental cells, or cells, e.g., stem cells, of another kind. In another embodiment, the conditioned medium comprises medium in which placental stem cells have been differentiated into an adult cell type. In another embodiment, the conditioned medium comprises medium in which placental perfusate cells and non-placental stem cells have been cultured.

5.7.2.4 Matrices Comprising Placental Cells

Further provided herein are matrices, hydrogels, scaffolds, and the like that comprise placental perfusate or placental perfusate cells.

Placental cells, e.g., perfusate or perfusate cells, can be seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental cells can be seeded are described in Hariri, U.S. Application Publication No. 2004/0048796.

Placental perfusate or placental perfusate cells can be suspended in a hydrogel solution suitable for, e.g., injection. Suitable hydrogels for such compositions include self-assembling peptides, such as RAD16. In one embodiment, a hydrogel solution comprising the cells can be allowed to harden, for instance in a mold, to form a matrix having cells dispersed therein for implantation. Placental perfusate or placental perfusate cells in such a matrix can also be cultured so that the cells are mitotically expanded prior to implantation. The hydrogel is, e.g., an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix is biodegradable.

In some embodiments, the formulation comprises an in situ polymerizable gel (see, e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

Placental perfusate or placental perfusate cells can be seeded onto a three-dimensional framework or scaffold and implanted in vivo. Such a framework can be implanted in combination with any one or more growth factors, cells, drugs or other components that stimulate tissue formation or otherwise enhance or improve the practice of the methods provided herein.

Examples of scaffolds that can be used in the methods provided herein include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

Placental perfusate or placental perfusate cells can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvern, Pa.). The framework can be a mixture, blend or composite of natural and/or synthetic materials.

In another embodiment, placental perfusate or placental perfusate cells can be seeded onto, or contacted with, a felt, which can be, e.g., composed of a multifilament yarn made from a bioabsorbable material such as PGA, PLA, PCL copolymers or blends, or hyaluronic acid.

Placental perfusate or placental perfusate cells can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1M acetic acid followed by incubation in polylysine, PBS, and/or collagen, prior to inoculation of the placental cells, e.g., placental perfusate cells, in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™. (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental perfusate or perfusate cells.

In one embodiment, placental stem cells are seeded onto, or contacted with, a suitable scaffold at about 0.5×10⁶ to about 8×10⁶ cells/mL.

5.7.3 Immortalized Placental Cell Lines

Mammalian placental cells can be conditionally immortalized by transfection with any suitable vector containing a growth-promoting gene, that is, a gene encoding a protein that, under appropriate conditions, promotes growth of the transfected cell, such that the production and/or activity of the growth-promoting protein is regulatable by an external factor. In a preferred embodiment the growth-promoting gene is an oncogene such as, but not limited to, v-myc, N-myc, c-myc, p53, SV40 large T antigen, polyoma large T antigen, E1a adenovirus or E7 protein of human papillomavirus.

External regulation of the growth-promoting protein can be achieved by placing the growth-promoting gene under the control of an externally-regulatable promoter, e.g., a promoter the activity of which can be controlled by, for example, modifying the temperature of the transfected cells or the composition of the medium in contact with the cells. in one embodiment, a tetracycline (tet)-controlled gene expression system can be employed (see Gossen et al., Proc. Natl. Acad. Sci. USA 89:5547-5551, 1992; Hoshimaru et al., Proc. Natl. Acad. Sci. USA 93:1518-1523, 1996). In the absence of tet, a tet-controlled transactivator (tTA) within this vector strongly activates transcription from ph_{CMV}*₋₁, a minimal promoter from human cytomegalovirus fused to tet operator sequences. tTA is a fusion protein of the repressor (tetR) of the transposon-10-derived tet resistance operon of Escherichia coli and the acidic domain of VP16 of herpes simplex virus. Low, non-toxic concentrations of tet (e.g., 0.01-1.0 μg/mL) almost completely abolish transactivation by tTA.

In one embodiment, the vector further contains a gene encoding a selectable marker, e.g., a protein that confers drug resistance. The bacterial neomycin resistance gene (neo^{R}) is one such marker that may be employed within the present methods. Cells carrying neo^{R} may be selected by means known to those of ordinary skill in the art, such as the addition of, e.g., 100-200 μg/mL G418 to the growth medium.

Transfection can be achieved by any of a variety of means known to those of ordinary skill in the art including, but not limited to, retroviral infection. In general, a cell culture may be transfected by incubation with a mixture of conditioned medium collected from the producer cell line for the vector and DMEM/F12 containing N2 supplements. For example, a placental cell culture prepared as described above may be infected after, e.g., five days in vitro by incubation for about 20 hours in one volume of conditioned medium and two volumes of DMEM/F12 containing N2 supplements. Transfected cells carrying a selectable marker may then be selected as described above.

Following transfection, cultures are passaged onto a surface that permits proliferation, e.g., allows at least about 30% of the cells to double in a 24 hour period. Preferably, the substrate is a polyornithine/laminin substrate, consisting of tissue culture plastic coated with polyornithine (10 μg/mL) and/or laminin (10 μg/mL), a polylysine/laminin substrate or a surface treated with fibronectin. Cultures are then fed every 3-4 days with growth medium, which may or may not be supplemented with one or more proliferation-enhancing factors. Proliferation-enhancing factors may be added to the growth medium when cultures are less than 50% confluent.

The conditionally-immortalized placental stem cell lines can be passaged using standard techniques, such as by trypsinization, when 80-95% confluent. Up to approximately the twentieth passage, it is, in some embodiments, beneficial to maintain selection (by, for example, the addition of G418 for cells containing a neomycin resistance gene). Cells may also be frozen in liquid nitrogen for long-term storage.

Clonal cell lines can be isolated from a conditionally-immortalized human placental stem cell line prepared as described above. In general, such clonal cell lines may be isolated using standard techniques, such as by limit dilution or using cloning rings, and expanded. Clonal cell lines may generally be fed and passaged as described above.

Conditionally-immortalized human placental stem cell lines, which may, but need not, be clonal, may generally be induced to differentiate by suppressing the production and/or activity of the growth-promoting protein under culture conditions that facilitate differentiation. For example, if the gene encoding the growth-promoting protein is under the control of an externally-regulatable promoter, the conditions, e.g., temperature or composition of medium, may be modified to suppress transcription of the growth-promoting gene. For the tetracycline-controlled gene expression system discussed above, differentiation can be achieved by the addition of tetracycline to suppress transcription of the growth-promoting gene. In general, 1 μg/mL tetracycline for 4-5 days is sufficient to initiate differentiation. To promote further differentiation, additional agents may be included in the growth medium.

5.7.4 Assays

Placental perfusate or placental perfusate cells can be used in assays to determine the influence of culture conditions, environmental factors, molecules (e.g., biomolecules, small inorganic molecules. etc.) and the like on stem cell proliferation, expansion, and/or differentiation, compared to placental perfusate or placental perfusate cells not exposed to such conditions.

In a preferred embodiment, placental perfusate or placental perfusate cells are assayed for changes in proliferation, expansion or differentiation upon contact with a molecule. For example, osteogenic differentiation can be assayed by monitoring alkaline phosphatase activity and/or calcium mineralization.

In one embodiment, for example, provided herein is a method of identifying a compound that modulates the proliferation of placental perfusate cells, comprising contacting said perfusate cells with said compound under conditions that allow proliferation, wherein if said compound causes a detectable change in proliferation of said cells compared to a plurality of said cells not contacted with said compound, said compound is identified as a compound that modulates proliferation of placental perfusate cells. In a specific embodiment, said compound is identified as an inhibitor of proliferation. In another specific embodiment, said compound is identified as an enhancer of proliferation.

In another embodiment, provided herein a method of identifying a compound that modulates the expansion of a plurality of placental cells, comprising contacting placental perfusate cells with said compound under conditions that allow expansion, wherein if said compound causes a detectable change in expansion of said cells compared to a plurality of cells not contacted with said compound, said compound is identified as a compound that modulates expansion of placental cells. In a specific embodiment, said compound is identified as an inhibitor of expansion. In another specific embodiment, said compound is identified as an enhancer of expansion.

In another embodiment, provided herein is a method of identifying a compound that modulates the differentiation of placental cells, e.g., placental perfusate cells, comprising contacting said cells with said compound under conditions that allow differentiation, wherein if said compound causes a detectable change in differentiation of said stem cells compared to a cell not contacted with said compound, said compound is identified as a compound that modulates proliferation of placental cells. In a specific embodiment, said compound is identified as an inhibitor of differentiation. In another specific embodiment, said compound is identified as an enhancer of differentiation.

### 6. EXAMPLES

The following examples are provided for illustration and are not to be construed to be limiting in any way. All references, whether patent references, literature references, or otherwise, cited herein are hereby incorporated by reference for all purposes.

### 6.1 Example 1

### Generation of Angiogenic Cells from Placental Perfusate Cells

Placental perfusate, obtained as described in Section 5.2, above, was depleted of erythrocytes and analyzed to determine the percentage of various mononuclear cell types. Table 1 details the cell types identified:

TABLE 1
Major nucleated cell populations in human
placental perfusate from a single placenta

| | | % |
|---|---|---|
| Cell Population | Surface Markers | 2 (n = 8) |
| NK cells | CD3⁻CD56⁺ | 6.45 |
| T cells | CD3⁺ | 21.6 |
| B cells | CD19⁺ | 6.97 |
| Endothelial progenitor cells | CD34⁺CD31⁺ | 1.69 |
| Neural progenitor cells | Nestin⁺ | 2.44 |
| Hematopoietic progenitor cells | CD34⁺ | 2.21 |
| Adherent placental stem cells | CD34⁻ CD117⁻ | 0.67 |
| | CD105⁺ CD44⁺ | |

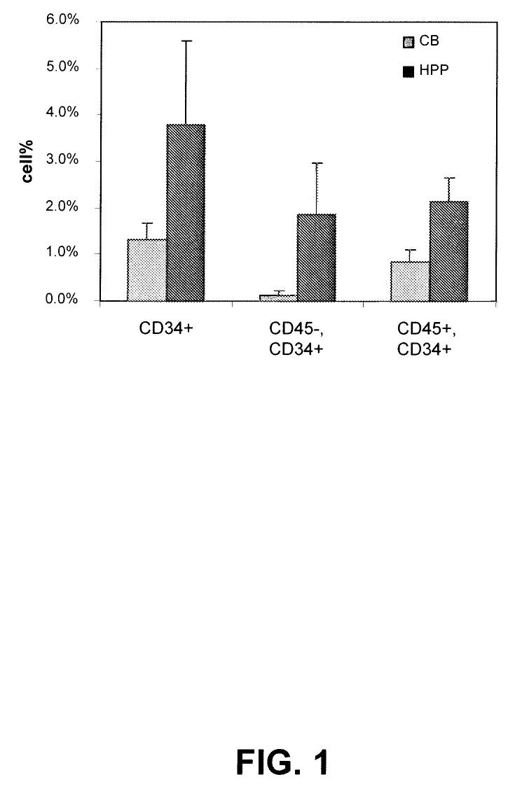

In a separate experiment, it was established that the CD34⁺ placental cells from human placental perfusate comprise a subpopulation of CD34⁺, CD45⁻ cells, which are present in a higher percentage for a given number of nucleated cells than in umbilical cord blood. See FIG. 1.

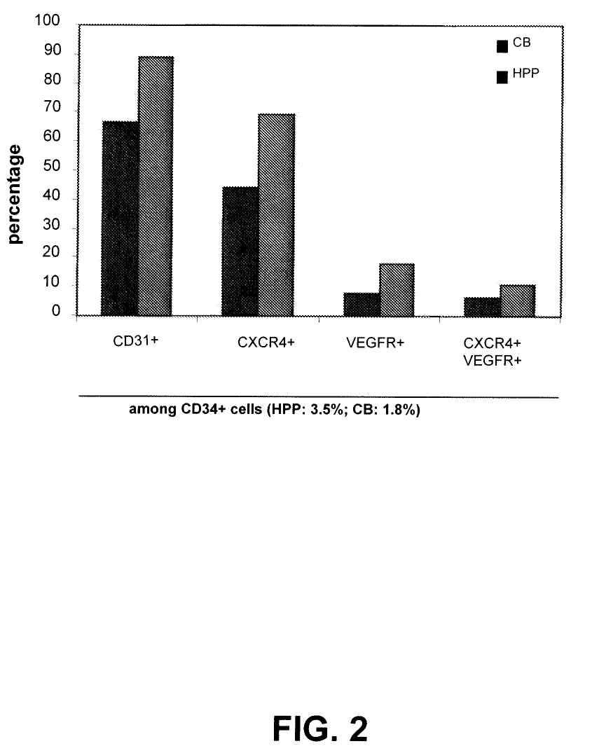

In another experiment, CD34⁺ cells from human placental perfusate were analyzed by flow cytometry to determine the percentage of cells expressing angiogenesis-related markers CD31, CXCR4 and VEGFR. A greater percentage of CD34⁺ cells from HPP expressed these markers than did CD34⁺ cells from umbilical cord blood. See FIG. 2.

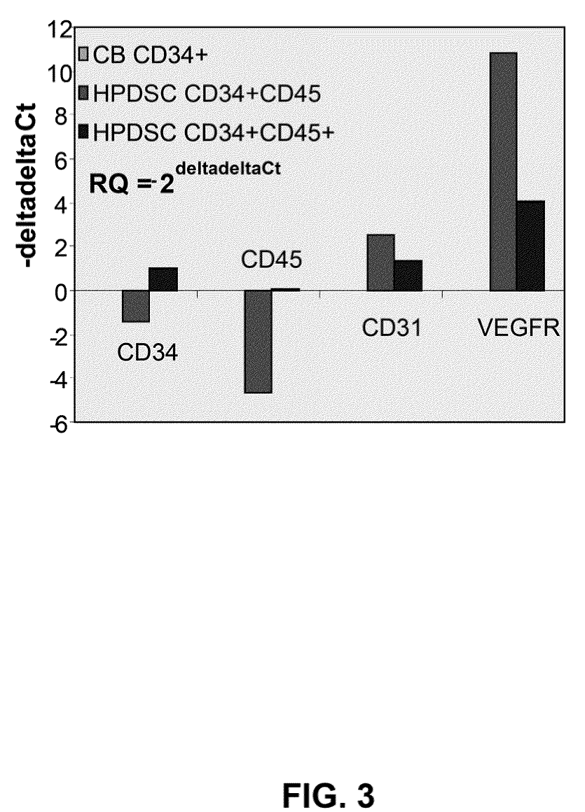

In another experiment, quantitative real-time PCR (qRT-PCR) was used to analyze gene expression in placental CD34⁺, CD45⁻ cells. CD34^{+CD}45⁻ and CD34^{+CD}45⁺ cell populations were isolated from the same human placental perfusate (HPP) by FACS ARIA (BD Biosciences) and were subjected to RNA preparation for qRT-PCR analysis of CD34, CD45, CD31 and VEGFR expression using an Applied Biosystems FAST 7900HT instrument and primer/probes. As shown in FIG. 3, both CD31 and VEGFR expression are higher in HPP CD34^{+CD}45 cells than in CD34^{+CD}45⁺ cells. These data suggested that HPP CD34⁺ cells are angiogenic, and in addition the angiogenic activity is more enriched in the CD34^{+CD}45⁻ population.

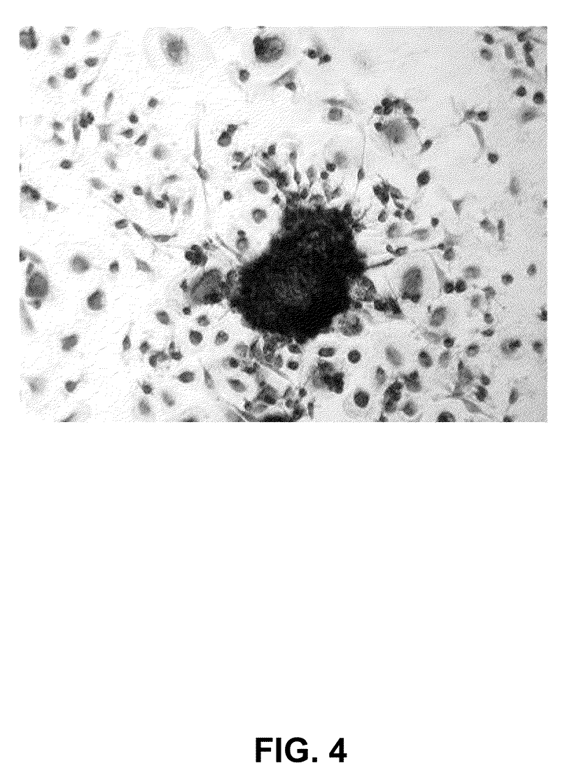

Angiogenic activity of HPP cells was determined using a CFU-Hill colony assay, which identifies precursors of endothelial cells. Mononuclear cells from human placental perfusate collected according to Example 6.3, above, were cultured in ENDOCULT® medium (StemCell Technologies, Inc.) for about 2 weeks. Cell cultures were then stained with Gill's Modified Hematoxylin stain to reveal CFU-Hill colonies. 0, 19, 7, 11 and 6 CFU-Hill colonies were identified per 10⁶ perfusate cells from five separate donors, respectively, in the assay. See FIG. 4. A separate assay confirmed the uptake of Dil-acLDL (diacetyl low density lipoprotein) by human placental perfusate-derived endothelial progenitor cells at day 7 of culture in ENDOCULT® medium.

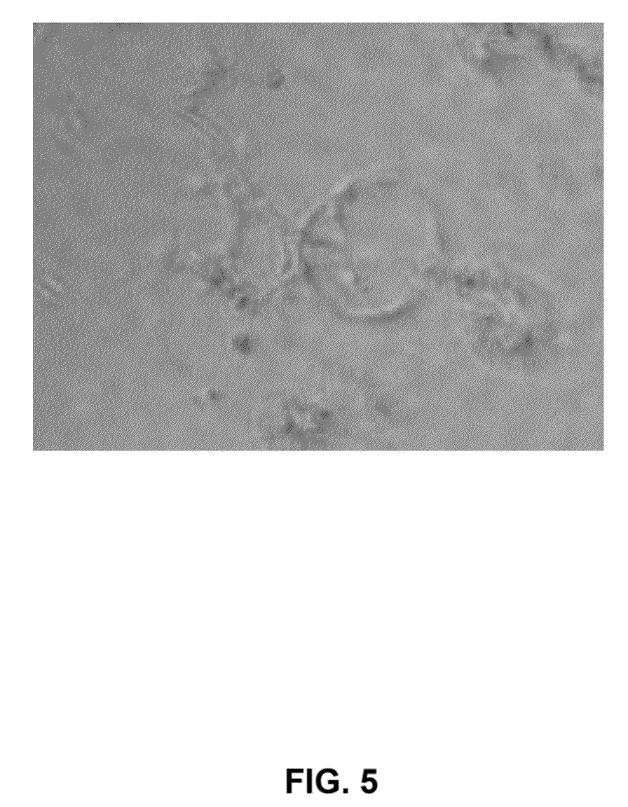

Human placental perfusate cells were also shown to develop vessels in culture. Human placental perfusate cells obtained according to Example 6.3, above, were cultured for 18-24 hours on ECMATRIX™ at about 10⁶ cells per well in a 96-well plate using In Vitro Angiogenesis Assay Kit (Chemicon cat# ECM625), in which the cells are cultured in the presence of TGF-β, FGF, plasminogen, tPA and matrix metalloproteases. The cells formed visible vessel structures after 24 hours. See FIG. 5. No significant tube formation was observed in cord blood cell culture under essentially the same conditions.

### 6.2 Example 2

### In Vivo Vessel Formation Using Placental Perfusate Cells

This example demonstrates that human placental perfusate cells, when administered into a rodent model, cause the formation of vasculature, as indicated by the formation of bone tissue.

Angiogenesis/vascularization is required for bone healing. See, for example, Matsumoto et al., Amer. J. Pathology 169:1440-1457 (2006) (adult human peripheral blood-derived CD34⁺ subpopulation has both angiogenic and osteogenic activity) and Matsumoto et al., Bone 2008 pages 1-6. The present experiment describes both in vivo bone-forming activity and angiogenesis by cells of human placental perfusate (HPP).

Bone-forming activity: Cranial defects (3 mm×5 mm) were created on each side of the calvaria of 6 week old athymic rats. Each left defect was treated with the Healos (DePuy Orthopaedics Inc., Warsaw, Ind.) carrier alone, while each right defect was treated either with a positive control (Healos+bone morphogenic protein 2 (BMP-2)), a negative control (empty defect), or with HPP+Healos. Eight animals were assigned to each treatment group. Rats were sacrificed 4 weeks following implantation. The calvariae were processed for histological analysis and tissue sections were stained with hematoxylin & eosin (H&E stain) according to the protocol in Table 2.

TABLE 2
H&E staining procedure

| Solution | Time | |
|---|---|---|
| Xylene substitute | 5 | min |
| Xylene substitute | 5 | min |
| 100% ethanol | 30 | s |
| 95% ethanol | 30 | s |
| 80% ethanol | 30 | s |
| 70% ethanol | 30 | s |
| Nanopure water | 20 | s |
| Hematoxylin | 10 | s |
| Run Tap water | 2 | min |
| Bluing reagent | 15-30 | s |
| 70% ethanol | 15-30 | s |
| Eosin Y | 2 | min |
| 80% ethanol | 30 | s |
| 95% ethanol | 30 | s |
| 95% ethanol | 30 | s |
| 100% ethanol | 30 | s |
| 100% ethanol | 30 s-1 min | |
| Xylene substitute | 5 | min |

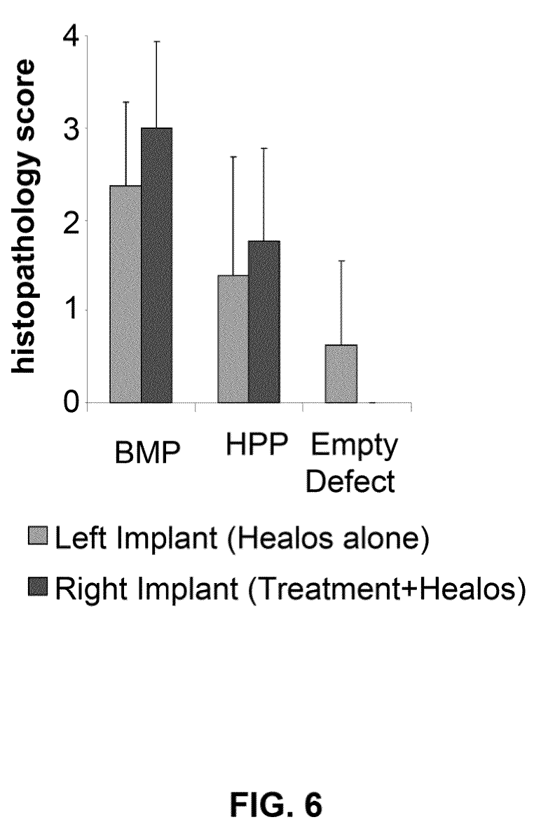

The amount of bone ingrowth into the defect was assessed by a 0 to 4 scoring system, with 4 as the largest amount (* p<0.05 compared to Healos alone (empty defect)) (See FIG. 6).

Angiogenesis: Vasculogenesis was demonstrated in explants in a group of animals subcutaneously implanted with HPP-seeded scaffold as compared to a group of animals implanted with scaffold alone.

### Material and Methods

Subcutaneous scaffold implants: Scaffolds were implanted into 6 week old (at study commencement) male Hsd:RH-Foxn1^{rnu} athymic rats. The rats were implanted with a circular diameter 5 mm scaffold (Vitoss Bone Graft Substitute, Orthovita) passively adsorbed with HPP at 5×10⁶ cells/mL for the test group. The control group was implanted with Vitoss alone. The rats were anesthetized and the implants placed subcutaneous in dorsal, ventral or thigh depending on group. On day 21 and day 42 post-surgery the selected rats were euthanized by CO₂ asphyxiation. The implants were collected and placed in 10% normal buffered formalin. After embedding in paraffin, 5 μm sections were processed for immunofluorescent staining.

Immunofluorescent Staining: To detect the transplanted human cells in the rat tissue, immunohistochemistry was performed (n=2) with human-specific CD34 endothelial cell marker mouse monoclonal antibody (clone QBEnd/10) IgG1 (Novocastra cat#NCL-L-END) at 1:50 dilution. Alpha smooth muscle actin (aSMA) mouse monoclonal (clone 1A4) from Dako cat#M0851 at 1:30 dilution was used to detect both human and rat smooth muscle cell. The secondary antibodies were as follows: Vector M.O.M. immunodetection kit fluorescein cat #FMK-2201 for CD34 and Alexa Flour 594-conjugated goat anti-mouse (Molecular Probes A21135) for aSMA. The positive control consisted of human tissue microarray with 34 different human tissues (Pantomics, Inc cat#MN0341) and the negative control was Max Array mouse tissue microarray slides (Zymed lab cat #75-2013).

Briefly, the slides were baked at 56° C. for 30 minutes (min), deparaffinized with xylene (three changes of 5 min each), rehydrated (passed through ethanol 100% to 70%), and blocked for endogenous peroxidase in 0.5% hydrogen peroxide in methanol at −20° C. Antigen retrieval was performed in microwaved 0.01 M Citrate buffer at pH 6.0 (two cycles, 10 min each). Avidin and biotin block was performed for 15 min. CD34 staining was performed according to the manufacturer's protocol using the Mouse-on-Mouse (M.O.M.) kit (Vector Laboratories). The second primary antibody (aSMA) was incubated overnight at 4° C., and the corresponding secondary antibody (AF594) was incubated for 20 min at room temperature. Between all steps the slides were washed with PBS three times each for 5 min. 4′,6-Diamidino-2-phenylindole (DAPI) solution was applied for 5 min for nuclear staining. The slides were coverslipped using an aqueous mounting medium.

Image Analysis: The slides were observed using a Nikon Eclipse E800 equipped with the appropriate epifluorescence filter sets and imaging software (N is Elements Basic Research). To evaluate angiogenesis, each slide was evaluated at a magnification of 20× in five different fields and the aSMA expression was assayed by measuring the percentage of expression in the field by the N is Elements software.

### Results

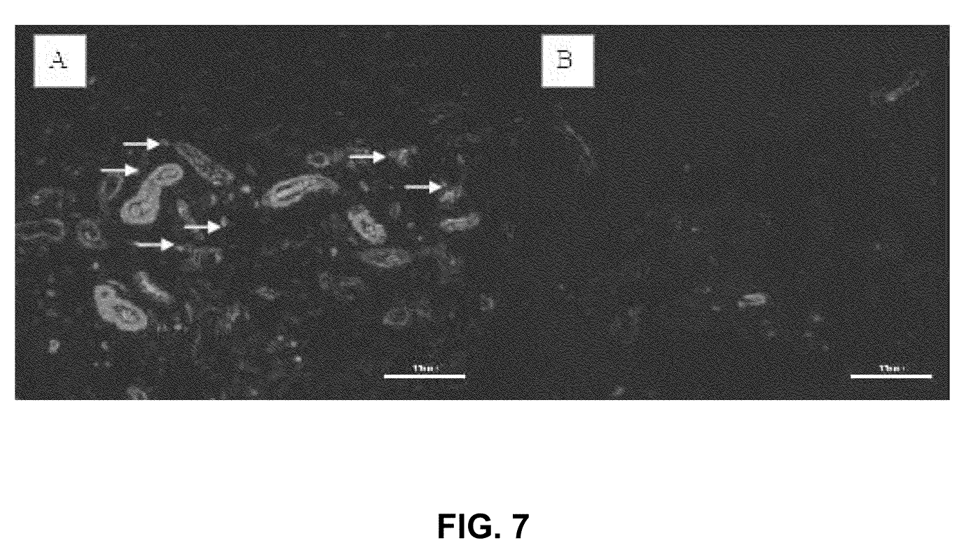

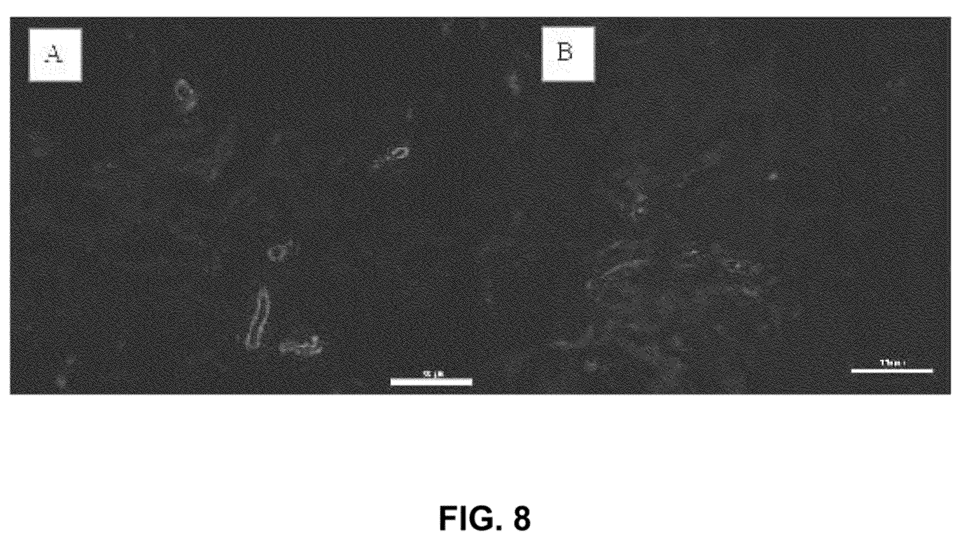

Enhancement of intrinsic angiogenesis in animals receiving HPP: Enhanced angiogenesis by paracrine effect of the transplanted cells on recipient were confirmed by positive immunostaining for aSMA and a lack of CD34 in endothelial cells. At the earlier timepoint (21 days) (n=2), more neovascularization was observed than the control group and there was no evidence of specific-human endothelial cell marker in these new vessels but some cells close to the vessels were stained for CD34 (FIG. 7). At the later timepoint (42 days), enhanced angiogenesis was observed but at a lower grade relative to the 21 day timepoint, and positive CD34 cells were not observed in the HPP group (FIG. 8).

The image analysis showed a statistically significant greater angiogenesis in the HPP group at 21 days compared to the control group (Vitoss alone) (p<0.01), no statistically significant difference was observed in the groups at 42 days (FIG. 9).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed:

1. A method of forming vessels from a population of placental perfusate cells, comprising contacting said population of cells with conditions that promote the formation of vessels.

2. The method of claim 1, wherein said population of placental perfusate cells is total nucleated cells from placental perfusate.

3. The method of claim 1, wherein said contacting comprises contacting said cells with VEGF (50 to 200 ng/mL), TGF-β (1 to 5 ng/mL), FGF (10 to 50 ng/mL) and one or more matrix metalloproteases (1 to 3 Unit/mL each).

4. The method of claim 1, wherein said contacting is performed in vitro.

5. The method of claim 1, wherein said contacting is performed in vivo.

6. The method of claim 1, wherein said population of placental perfusate cells comprises placental perfusate cells isolated from perfusion of a single placenta.

7. The method of claim 6, wherein said placental perfusate cells are CD34⁺ cells.

8. The method of claim 7, wherein said CD34⁺ cells are CD34^{+CD}45⁻ cells.

9. The method of claim 6 or claim 7, wherein said CD34⁺ cells or CD34^{+CD}45⁻ cells express a higher level of at least one of CD31, CXCR4 or VEGFR than an equivalent number of CD34⁺ cells from umbilical cord blood.

10. The method of claim 1, wherein said population of placental perfusate cells comprises isolated CD34⁺ cells not isolated from said perfusate.

11. The method of claim 10, wherein said CD34⁺ cells are isolated from placenta.

12. The method of claim 10, wherein said CD34⁺ cells are isolated from umbilical cord blood, placental blood, peripheral blood, or bone marrow.

13. The method of claim 10, wherein said CD34⁺ cells express a higher level of CD31, CXCR4 or VEGFR than an equivalent number of CD34⁺ cells from umbilical cord blood.

14. The method of claim 10, wherein said CD34⁺ cells are CD34⁺, CD45⁻ cells.

15. A method of treating an individual having a cardiac or vascular disease, disorder, condition or insufficiency, comprising administering human placental perfusate or human placental perfusate cells to said individual in an amount sufficient to treat said disease, disorder, condition or insufficiency.

16. The method of claim 15, wherein said disease, disorder, condition or insufficiency is peripheral vascular disease, acute or chronic myocardial infarct, cardiomyopathy, congestive or chronic heart failure, cardiovascular ischemia, hypertensive pulmonary vascular disease, peripheral arterial disease, or rheumatic heart disease.

17. The method of claim 15, wherein said placental perfusate cells are total nucleated cells from placental perfusate.

18. The method of claim 15, wherein said population of placental perfusate cells comprises placental perfusate cells isolated from perfusion of a single placenta.

19. The method of claim 18, wherein said placental perfusate cells are CD34⁺ cells.

20. The method of claim 19, wherein said CD34⁺ cells are CD34^{+CD}45⁻ cells.

21. The method of claim 19 or claim 20, wherein said CD34⁺ cells or CD34^{+CD}45⁻ cells express a higher level of at least one of CD31, CXCR4 or VEGFR than an equivalent number of CD34⁺ cells from umbilical cord blood.

22. The method of claim 15, wherein said population of placental perfusate cells comprises isolated CD34⁺ cells not isolated from said perfusate.

23. The method of claim 22, wherein said CD34⁺ cells are isolated from placenta.

24. The method of claim 22, wherein said CD34⁺ cells are isolated from umbilical cord blood, placental blood, peripheral blood, or bone marrow.

25. The method of claim 22, wherein said CD34⁺ cells express a higher level of CD31, CXCR4 or VEGFR than an equivalent number of CD34⁺ cells from umbilical cord blood.

26. The method of claim 15, wherein said placental perfusate cells are administered on a scaffold or matrix.

27. The method of claim 15, wherein said placental perfusate cells are administered intravenously.
